(19) United States
(12) Patent Application Publication
(10) Pub. No.: US 2026/0224226 A1
(43) Pub. Date: Aug. 6, 2026

(54) PNEUMATIC EMERGENCY FIELD TOURNIQUET DEVICE AND METHODS FOR USING SAME

(71) Applicant: Nathan Williams, Atlanta, GA (US)

(72) Inventor: Nathan Williams, Atlanta, GA (US)

(21) Appl. No.: 19/631,384

(22) Filed: Mar. 27, 2026

Related U.S. Application Data
(63) Continuation-in-part of application No. 16/785,534, filed on Feb. 7, 2020.
(60) Provisional application No. 62/802,348, filed on Feb. 7, 2019.

(51) Int. Cl. A61B 17/135

(52) U.S. Cl. CPC A61B 17/135

(57) ABSTRACT

A pneumatic tourniquet system includes a pressurized gas source, pressure regulator, and inflatable bladder integrated within a multi-layer band assembly. The system includes a spring steel encircling member positioned between structural layers that provides constraint preventing radial bladder expansion during gas inflation. A dual-rate valve system enables toggling between different inflation rates for controlled pressure application. The spring steel member has a one-way directional potential energy driven state transition from an extended state to a coiled state that automatically positions the bladder for arterial compression. The integrated pneumatic and mechanical elements direct gas pressure inward for vascular compression while eliminating external stiffening components. A fastening mechanism secures the band assembly around a limb. The system provides hemostatic pressure control through coordinated pneumatic inflation and mechanical constraint forces.

### RELATED APPLICATIONS

This application is a Continuation-In-Part application under 35 U.S.C. § 120 of U.S. application Ser. No. 16/785,534 filed on Feb. 7, 2020, which claims the benefit under 35 U.S.C. § 119 (e) of U.S. Provisional Application No. 62/802,348 filed on Feb. 7, 2019.

It is intended that the referenced application may be applicable to the concepts and embodiments disclosed herein, even if such concepts and embodiments are disclosed in the referenced application with different limitations and configurations and described using different examples and terminology.

### FIELD OF DISCLOSURE

The present disclosure generally relates to emergency medical tourniquets and pneumatic hemostatic devices for use in pre-hospital and field emergency situations; namely, tourniquets with a flexible encircling member with a pneumatic means for applying local pressure for hemorrhage control outside of hospital settings; and methods for using same.

### BACKGROUND OF THE DISCLOSURE

In certain serious medical situations, specific medical intervention may be necessary to constrict or control blood flow to one or more limbs. For example, if blood flow is not controlled or constricted, severe blood loss from that limb can result in the loss of that limb or life. The conventional method for restricting blood flow involves using a primitive constricting or compressing device to control arterial and venous blood flow to a limb or extremity for a period of time. This often causes problems because the conventional strategy does not allow for a regulated release of pressure which can be essential in preserving the limb. Further, basic medical knowledge and training may be necessary for proper application of this medical intervention to achieve the most desirable medical outcomes. Moreover, most current medical interventions may require two hands to effectively execute. Other forms of current medical interventions also can take longer to administer than is desirable. For example, in combat situations and traumatic injuries, improper and/or delayed medical intervention may result in furthering the injury.

There are many technical challenges in emergency hemorrhage control that may occur prior to a hospital visit in field emergency situations. Traumatic hemorrhage remains a leading cause of preventable death in both civilian and military trauma scenarios. Current tourniquet devices present multiple technical limitations that restrict their effectiveness in emergency applications outside controlled medical environments.

A fundamental technical problem exists in enabling injured individuals to self-apply tourniquets using a single hand. When an individual sustains a traumatic injury to a limb, the dominant hand may be compromised or the injured limb may be the dominant arm itself. Conventional tourniquet designs require two-handed operation, creating an insurmountable barrier to self-application. An injured person attempting to control hemorrhage from their own dominant arm cannot physically manipulate traditional tourniquets that demand bimanual coordination.

Time represents a critical factor in traumatic hemorrhage scenarios. Exsanguination from major limb trauma can result in death within minutes. Current tourniquet systems require application times ranging from thirty seconds to multiple minutes. Each second of delay increases blood loss volume and decreases survival probability. Technical solutions requiring complex multi-step procedures or precise manual adjustments consume precious time during which continued hemorrhage compromises the victim.

Emergency situations occur in unpredictable environments without access to medical equipment or trained personnel. A construction worker suffers a severe laceration from machinery at a remote job site. A hiker encounters a traumatic injury miles from the nearest road. A victim of a mass casualty incident requires immediate hemorrhage control before first responders arrive. These scenarios present technical challenges distinct from controlled hospital settings.

The absence of medical expertise creates problems for devices requiring anatomical knowledge or pressure calibration skills. An untrained bystander cannot accurately determine appropriate occlusion pressures or identify proper placement locations. Devices demanding such expertise remain ineffective in the hands of civilians who encounter emergency situations.

Technical challenges intensify in remote locations where professional medical assistance remains hours away. A hunter sustains a severe leg injury in wilderness terrain. A sailor experiences traumatic hemorrhage aboard a vessel far from shore. A soldier encounters combat trauma in an austere environment. These situations demand tourniquet solutions that function reliably without external support infrastructure.

Current devices requiring external power sources, monitoring equipment, or pneumatic systems become non-functional in field conditions. Battery depletion renders electronic systems inoperative. Pneumatic connections and external inflation devices add bulk and complexity incompatible with emergency response kits or individual first aid supplies.

Tourniquets must achieve sufficient pressure to occlude arterial blood flow while avoiding excessive pressure that damages tissue. Manual tourniquet systems depend entirely on user-applied force, resulting in highly variable and unpredictable pressure levels. An exhausted or injured person lacks the physical strength to generate adequate occlusive pressure. Conversely, panic-driven over-tightening creates dangerously high pressures that cause nerve damage and tissue necrosis.

The human element introduces inconsistency that technical solutions must address.

Different users applying the same device to similar injuries generate pressure variations spanning hundreds of millimeters of mercury. This variability produces either ineffective hemorrhage control or iatrogenic injury from excessive compression.

Emergency scenarios rarely permit victims to remain stationary. Evacuation from danger zones requires movement. Self-rescue from hazardous locations demands mobility. Current tourniquet designs that function adequately under static conditions fail during dynamic activities. Narrow bands migrate along the limb during movement. Rigid components interfere with joint articulation. Bulky assemblies catch on obstacles or equipment.

A victim attempting to exit a collapsed structure while wearing a conventional tourniquet encounters mechanical interference. The device shifts position, losing effectiveness. Protrusions snag on debris. The victim must choose between maintaining hemorrhage control and achieving mobility to reach safety.

Limb muscles undergo cyclical contraction and relaxation during movement or involuntary spasm. These dimensional changes alter the relationship between tourniquet and limb. Rigid inelastic bands cannot accommodate circumferential expansion during muscle contraction. The limb presses against an unyielding barrier, creating localized pressure spikes and pain. Alternatively, elastic bands that stretch excessively lose occlusive pressure during limb expansion.

The technical challenge requires maintaining adequate occlusive pressure throughout the full range of limb dimensional changes without creating pressure spikes that cause pain or tissue damage. Current solutions fail to achieve this balance.

Effective tourniquet application requires training and practice. Medical professionals undergo extensive instruction in proper technique. Military personnel receive repeated hands-on training. This training burden prevents widespread tourniquet availability to civilian populations. A device requiring hours of instruction cannot be deployed as a public safety tool comparable to automated external defibrillators.

Complex application procedures overwhelm users under stress. Cognitive function deteriorates during emergencies. Panic impairs fine motor control. A tourniquet design demanding precise sequential steps or careful adjustment becomes unusable when users most need it.

While tourniquets must restrict blood flow, complete arterial occlusion carries significant risks. Prolonged total ischemia results in irreversible tissue damage. Nerve injury occurs from excessive pressure. The technical challenge involves restricting venous return and reducing arterial flow without completely occluding circulation. Current devices designed for surgical applications intentionally achieve complete occlusion, making them unsuitable for extended field use where gradual reperfusion may be necessary.

Eventually, tourniquets must be removed or pressure must be reduced. Sudden complete pressure release causes reperfusion injury. Accumulated metabolites and inflammatory mediators flood the systemic circulation. Compartment syndrome develops from rapid fluid shifts. Current field tourniquets provide only binary states: fully applied or completely removed. The technical challenge requires controlled, gradual pressure reduction to minimize reperfusion complications.

Tourniquets necessarily cause discomfort. However, excessive pain prevents proper application or causes premature removal. Narrow bands concentrate pressure over small areas, creating intense focal pain. Rigid components dig into tissue during muscle movement. Users experiencing severe pain may loosen or remove the device prematurely, compromising hemorrhage control.

The technical challenge requires distributing compressive forces to minimize pain while maintaining effective restriction. Current solutions prioritize mechanical simplicity over user comfort, limiting their practical utility in conscious persons who must tolerate the device during extended periods.

Accordingly, existing tourniquet devices present limitations in emergency hemorrhage control applications. Conventional tourniquet systems require two-handed operation for proper application. Traditional windlass-based tourniquets demand extended application times ranging from thirty seconds to several minutes. Manual tightening mechanisms depend on user physical strength to achieve adequate occlusive pressure. Current devices provide inconsistent pressure levels across different users and application scenarios. Existing tourniquets lack automated pressure regulation systems for field deployment. Conventional designs cannot be effectively applied by an injured person to their own limb using only their non-injured hand. Standard tourniquet removal procedures require cutting or manual loosening without controlled pressure release capability. Existing devices do not provide integration with external pneumatic systems for gradual deflation. Current tourniquet technologies require extensive training for proper application technique. Available devices lack intuitive deployment mechanisms suitable for untrained civilian users in emergency situations.

### BRIEF OVERVIEW

In various aspects, the invention relates to devices, systems, and methods for restricting local blood flow, such as controlling blood flow to an injured limb. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

A pneumatic tourniquet system may comprise a pressurized gas source containing compressed gas. The system may comprise a pressure regulator configured to reduce gas pressure from the pressurized gas source to a working pressure. An inflatable bladder may be configured to receive gas from the pressure regulator. A gas delivery conduit may provide fluid communication between the pressure regulator and the inflatable bladder. A band assembly may be configured to position the inflatable bladder against a limb for hemostatic pressure application. A fastening mechanism may be configured to secure the band assembly around the limb. The pneumatic system may deliver gas to the inflatable bladder to provide controlled arterial restriction.

A multi-layer band assembly for a tourniquet device may comprise a top layer configured to provide structural protection. A middle layer may be disposed beneath the top layer. A spring steel encircling member may be positioned between the top layer and middle layer. A bottom layer may be disposed beneath the middle layer. An inflatable chamber may be positioned between the middle layer and bottom layer. A fastening component may be configured to secure the band assembly around a limb. The spring steel encircling member may have a one-way directional potential energy driven state transition from an extended state to a coiled state.

An integrated pneumatic-mechanical tourniquet system may comprise a pressurized gas source containing compressed gas. A pressure regulator may be configured to reduce gas pressure from the pressurized gas source. A multi-layer band assembly may include a spring steel encircling member and an inflatable bladder. A gas delivery system may provide fluid communication between the pressure regulator and the inflatable bladder. The spring steel encircling member may provide structural constraint that prevents radial bladder expansion during pneumatic inflation, directing gas pressure inward for vascular compression.

In accordance with the purposes of the disclosure, as embodied and broadly described herein, the disclosure, in one aspect, relates to an improved tourniquet device and system, such as, for example a quick tourniquet. In further aspects, the disclosure relates to a slap-on tourniquet. In another exemplary aspect, the disclosure relates to a fast-acting precision tourniquet. In further aspects, the disclosure also relates to an accelerated tourniquet method.

In yet another aspect, disclosed herein are devices and systems for using a fast-acting tourniquet device. In further aspects, the disclosure provides a device for controlling local blood flow, the device comprising: a pressurized gas source; a switch in operable communication with the pressurized gas source, the switch configured to selectively release or dispense a predetermined volume of gas in a gas stream having a predetermined flow rate and a predetermined flow path; and a band comprising a bendable encircling member and an air compartment [or air bladder], the compartment in operable communication with the gas stream; the encircling member having an extended state and coiled state configured to circumferentially couple around a portion of a user's body; and a fastening means configured to releasably secure the band around the portion of the user's body; wherein the gas stream is configured to fill at least a portion of the compartment effective to cause the band to exert a predetermined level of pressure against the portion of the user's body effective to reduce local blood flow.

In further aspects, the disclosure provides a fast acting tourniquet device, comprising: a slap band having a spring mechanism wherein the slap band is the medium of application of the device to a limb member; an air bladder configured to provide inflation pressure; an air canister chamber configured to trigger activation of the inflation circuit of the air bladder; a latch configured to close the air bladder around the limb member; and an air valve configured to control release of the device.

In yet another aspect, disclosed herein are methods of using the disclosed fast-acting tourniquet devices and systems. In further aspects, disclosed herein is a method, comprising: applying a slap band of a tourniquet device to a limb member; securing the tourniquet device having an air bladder around the limb member using a latch or clasp; triggering inflation circuit of the air bladder using an air canister; and controlling a release of the tourniquet device using an air valve.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 shows a depiction of a Tourniquet Device in accordance with an exemplary embodiment of the present disclosure.

FIGS. 2A-2B show depictions of a band assembly of a Tourniquet Device in accordance with an exemplary embodiment of the present disclosure.

FIGS. 3A-3C show depictions of a band assembly of a Tourniquet Device in accordance with an exemplary embodiment of the present disclosure.

FIGS. 4A-4B show depictions of a band assembly of a Tourniquet Device in accordance with an exemplary embodiment of the present disclosure.

FIG. 5 shows a depiction of an air canister assembly of a Tourniquet Device in accordance with an exemplary embodiment of the present disclosure.

FIG. 6 shows a depiction of an air canister assembly of a Tourniquet Device in accordance with an exemplary embodiment of the present disclosure.

FIG. 7 shows a depiction of an air canister assembly of a Tourniquet Device in accordance with an exemplary embodiment of the present disclosure.

FIG. 8 shows a depiction of an air canister assembly of a Tourniquet Device in accordance with an exemplary embodiment of the present disclosure.

FIGS. 9A-9B show depictions of an air canister assembly of a Tourniquet Device in accordance with an exemplary embodiment of the present disclosure.

FIGS. 10A-10B show depiction of an air canister assembly of a Tourniquet Device in accordance with an exemplary embodiment of the present disclosure.

FIG. 11 shows a depiction of a method for using a Tourniquet Device on a limb in accordance with an exemplary embodiment of the present disclosure.

FIG. 12 shows a depiction of a method for controlling blood flow to a limb using a Tourniquet Device in accordance with an exemplary embodiment of the present disclosure.

FIG. 13 is a flow chart of a method for controlling blood flow to a limb using a Tourniquet Device in accordance with an exemplary embodiment of the present disclosure.

FIG. 14 is an exploded, cross-sectional view of a band assembly of a Tourniquet Device in accordance with an exemplary embodiment of the present disclosure.

### DETAILED DESCRIPTION OF THE DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Regarding applicability of 35 U.S.C. § 112, 16, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of tourniquet devices, embodiments of the present disclosure are not limited to use only in this context. The present disclosure can be understood more readily by reference to the following detailed description of the disclosure and the Examples included therein.

Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

The pneumatic tourniquet system disclosed herein represents an advancement in pressure application instruments, specifically addressing the technical challenges of controlled gas delivery and pressure regulation in emergency hemostatic devices. The precision valve mechanisms, pressure monitoring systems, and gas flow control elements are critical components that distinguish this invention from simple mechanical tourniquets. The dual-rate inflation capability and pressure relief systems require specialized pneumatic engineering typically found in advanced instrumentation rather than basic mechanical devices.

The disclosed tourniquet devices and systems address multiple technical challenges that arise in emergency hemorrhage control situations. These challenges span mechanical, physiological, operational, and clinical domains. The primary use case focuses on traumatic limb injuries requiring rapid blood flow occlusion to prevent exsanguination, with particular emphasis on self-administration scenarios where the injured person must apply the tourniquet to their own limb using only one hand.

Conventional tourniquet devices may require extended application times that can prove fatal in severe hemorrhage situations. Traditional windlass-style tourniquets may necessitate 30 seconds to several minutes for proper application, depending on user training and physical capability. This time delay may allow continued blood loss at rates of 500 to 1500 milliliters per minute in cases of major arterial injury. The disclosed device addresses this problem through a dual-mechanism approach. The spring steel encircling member may enable initial band positioning in less than one second through potential energy release upon contact with the limb. The pneumatic inflation system may then achieve occlusive pressure within 2 to 5 seconds following activation. The combined application time may be reduced to under 10 seconds, representing a three-fold to sixty-fold improvement over conventional devices.

Existing tourniquet designs may require two-handed operation for effective application. A user may need one hand to position the band and a second hand to operate a windlass mechanism or tightening system. This requirement may render self-application impossible when the injured limb is an arm, as the injured arm cannot assist in tourniquet placement. The disclosed slap band mechanism may solve this problem by eliminating the need for manual wrapping. The spring steel member may automatically coil around the limb upon striking the band against the limb surface. The fastening mechanism may be engaged with the same hand through a pull tab attached to a latch or hook component. The inflation activation may similarly require only a single pulling motion on an activation element. Each step may be completed sequentially using only the uninjured hand.

Manual tourniquet tightening may produce highly variable occlusive pressures depending on user strength, training, and physical condition. Studies may show pressure variations ranging from 50 millimeters of mercury to over 400 millimeters of mercury with conventional devices. Insufficient pressure may fail to occlude arterial blood flow, while excessive pressure may cause unnecessary tissue damage, nerve injury, and pain. The pneumatic inflation system may address this problem through predetermined pressure delivery. The compressed gas cartridge may contain a fixed volume of gas at a known pressure. The air bladder may be sized to achieve a target pressure of 200 to 300 millimeters of mercury when filled with the predetermined gas volume. This mechanized pressurization may eliminate user strength as a variable and may ensure consistent occlusive pressure across all applications.

Narrow tourniquet bands and windlass mechanisms may concentrate pressure over small contact areas, typically 1 to 2 inches in width. This pressure concentration may cause severe pain, soft tissue damage, and nerve compression injuries. The pain may be so severe that conscious users may request premature tourniquet removal despite continued hemorrhage risk. The disclosed air bladder may distribute pressure over a substantially larger surface area, typically 3 to 6 inches in width and 12 to 24 inches in circumference. The bladder may conform to the limb contour during inflation, creating uniform pressure distribution. This larger contact area may reduce peak pressure points while maintaining adequate total compressive force for vascular occlusion. The distributed pressure may significantly reduce pain perception and tissue trauma.

Rapid tourniquet removal may cause reperfusion injury and compartment syndrome. When a tourniquet is suddenly released after prolonged application, accumulated metabolic waste products and inflammatory mediators may flood into systemic circulation. Simultaneously, rapid restoration of blood flow to ischemic tissue may cause reactive hyperemia and edema formation within fascial compartments. Conventional tourniquets may offer no mechanism for controlled, gradual pressure reduction. The disclosed valve system may address this problem by enabling adjustable deflation rates. The valve may be partially opened to allow gas escape at controlled rates, such as 10 to 20 millimeters of mercury per minute. This gradual pressure reduction may allow staged reperfusion, giving tissue time to accommodate restored blood flow and preventing sudden metabolite washout.

The synergistic combination of the slap band mechanism and pneumatic inflation system provides unexpected technical advantages over conventional tourniquet designs. The spring steel encircling member may provide initial circumferential constraint that prevents the air bladder from expanding radially outward during inflation, a common problem in prior art pneumatic tourniquets that require external stiffening elements. This constraint may direct all pneumatic pressure inward toward the limb, maximizing occlusive efficiency while minimizing required inflation pressure. The slap band's automatic conforming action may create uniform contact pressure distribution around irregular limb geometries, eliminating pressure concentration points that can cause tissue damage or inadequate vascular compression.

The technical synergy extends to pressure maintenance during muscle contraction. When underlying muscle tissue expands during contraction, conventional tourniquets may experience significant pressure loss as the rigid band cannot accommodate tissue volume changes. The disclosed slap band mechanism may provide controlled elastic response that maintains consistent vascular compression while allowing limited tissue expansion. This spring-like behavior may prevent the pressure spikes and drops that can cause user discomfort and compromise hemostatic effectiveness. The combination enables sustained arterial occlusion pressures of 200-300 mmHg with safety margins of 30-50 mmHg above systolic pressure, providing reliable hemostasis without excessive tissue trauma.

The air compartment construction may utilize heat-welded or adhesive-bonded seams to create a leak-proof inflatable chamber between the middle and bottom layers. The air compartment may be formed by bonding the middle layer to the bottom layer around the perimeter, leaving an inlet port for gas connection. The bonding may be achieved through ultrasonic welding, heat sealing, or structural adhesives that create seam strengths of 20 to 50 pounds per linear inch. The seam width may be 0.125 to 0.500 inches to provide adequate bonding area while minimizing bulk.

The gas inlet connection may comprise a threaded fitting, push-connect coupling, or barbed hose connector that provides secure attachment to the gas delivery system. The inlet may include a one-way check valve that prevents gas backflow when the canister is disconnected. The check valve may be a spring-loaded ball valve, reed valve, or diaphragm valve that opens at 2 to 10 psi inlet pressure and seals against reverse flow. The inlet fitting may be reinforced with a backing plate or grommet to prevent tearing of the air compartment material under connection forces.

The air compartment may include internal baffles or channels that promote uniform pressure distribution across the contact area. The baffles may be formed by selective bonding of the middle and bottom layers at predetermined locations, creating a grid pattern or parallel channels that prevent air from concentrating in localized areas. The baffle spacing may be 0.5 to 2.0 inches, creating individual pressure cells that conform independently to limb contours. This compartmentalized design may prevent pressure migration and maintain consistent contact pressure even when the limb moves or changes position.

The fluid communication system may include flexible tubing and quick-disconnect fittings that allow the gas canister to be positioned away from the band assembly. The tubing may be reinforced polymer hose with an internal diameter of 0.125 to 0.250 inches and a working pressure rating of 200 to 500 psi. The tubing length may be 6 to 24 inches, providing sufficient reach for canister positioning while minimizing bulk and weight. The quick-disconnect fittings may be push-to-connect or threaded couplings that allow rapid attachment and removal of the gas canister without tools or gas loss.

The mechanical occlusion forces generated by the spring steel constraint system may operate independently of gravitational and atmospheric conditions, enabling effective tourniquet function in extreme environments where conventional devices may fail. Unlike purely pneumatic tourniquets that rely on atmospheric pressure differentials for proper operation, the disclosed system's mechanical constraint forces are generated by stored elastic energy in the spring steel member that remains constant regardless of external atmospheric pressure. The spring steel member may maintain its structural properties and constraint forces at atmospheric pressures ranging from near-vacuum conditions (0.1 psia) to high-pressure environments (50+ psia), making the device suitable for use in depressurized aircraft cabins, high-altitude mountaineering, underwater diving operations, submarine environments, and deep mining applications.

The gravitational independence of the mechanical system may enable tourniquet function in variable gravity environments including microgravity conditions. The spring steel constraint mechanism relies on elastic deformation forces rather than gravitational effects, allowing consistent operation in reduced gravity environments such as space stations, lunar surfaces, planetary colonization activities where different gravities may be experienced, or other planetary bodies. The pneumatic inflation system may require minor pressure adjustments for different atmospheric conditions, but the fundamental mechanical constraint and pressure distribution functions remain unaffected by gravitational variations. This environmental independence may be particularly valuable for emergency medical applications in aerospace, deep-sea exploration, extreme altitude operations, future space exploration missions, and/or the like, where conventional medical devices may experience reduced effectiveness due to atmospheric or gravitational variations.

The system's dual-mode operation may provide redundant hemostatic capability even if atmospheric pressure changes affect pneumatic performance. In extreme low-pressure environments where gas expansion could reduce pneumatic effectiveness, the mechanical spring steel constraint may continue to provide baseline compression forces (in some cases, forces sufficient for emergency hemostatic control). Conversely, in high-pressure environments where pneumatic inflation might be enhanced, the mechanical constraint prevents over-pressurization and maintains safe pressure limits. This environmental adaptability may extend the operational envelope of the tourniquet system beyond conventional atmospheric and gravitational conditions encountered in standard medical applications.

The pressurized gas source may be configured with different canister specifications optimized for specific atmospheric pressure environments to maintain consistent hemostatic effectiveness across varying ambient conditions. For standard atmospheric pressure applications (e.g., 14.7 psia at sea level), the gas canister may contain compressed air or nitrogen at 800-1200 psi. For high-altitude applications (e.g., where ambient pressure may be reduced to 8-12 psia), the canister may contain gas at higher pressures of 1200-1800 psi to compensate for the reduced pressure differential and maintain effective bladder inflation. For underwater or hyperbaric applications where ambient pressure may exceed 20-100 psia, the canister may contain gas at correspondingly higher pressures of 1500-3000 psi to overcome the increased external pressure resistance.

The pressure regulator system may include adjustable or interchangeable components that accommodate different canister pressures while maintaining consistent output pressure to the bladder. The regulator may include selectable spring elements or adjustable spring preload mechanisms that can be configured for different input pressure ranges. Alternative embodiments may include multiple pressure regulators or a variable-output regulator with manual or automatic adjustment capabilities based on ambient pressure sensing. The regulator output pressure may be maintained at 50-150 psi above ambient pressure regardless of the input canister pressure, ensuring consistent bladder inflation characteristics across different environmental conditions.

Canister selection protocols may be established based on anticipated operating environments and ambient pressure conditions. Standard canisters rated for sea-level operations may be marked with color coding or labeling indicating their pressure range and suitable altitude limits. High-pressure canisters for extreme altitude or aerospace applications may include enhanced safety features such as burst discs rated for higher pressures and reinforced canister construction. Underwater or hyperbaric canisters may include corrosion-resistant materials and sealing systems designed for high-pressure differential environments. The canister selection may be based on anticipated ambient pressure ranges, with safety margins of 200-500 psi above the minimum required differential pressure for effective bladder inflation.

In some embodiments, the system may include ambient pressure compensation mechanisms that automatically adjust gas delivery based on environmental conditions. An integrated barometric pressure sensor may monitor ambient conditions and provide feedback to adjust regulator output pressure or valve timing to maintain consistent hemostatic effectiveness. The compensation system may include lookup tables and/or algorithmic controls that modify gas delivery parameters based on measured ambient pressure, ensuring that the bladder achieves target compression pressures of 200-250 mmHg regardless of environmental conditions. This adaptive capability may enable a single device configuration to operate effectively across altitude ranges from below sea level to 30,000+ feet elevation without manual adjustment or canister replacement.

An intelligent pressure control embodiment may include an integrated barometric pressure sensor and artificial intelligence system that automatically determines optimal inflation pressure based on ambient atmospheric conditions and continuously adjusts tourniquet pressure to maintain hemostatic effectiveness. The barometric sensor may comprise a miniature MEMS pressure transducer capable of measuring ambient atmospheric pressure with accuracy of ±0.1 psia over a range from 0.5 to 50 psia. The sensor may be integrated into the gas canister assembly or control unit housing, with environmental protection to prevent contamination while allowing pressure equalization with ambient conditions.

The artificial intelligence system may comprise a microprocessor or microcontroller with embedded machine learning algorithms trained on hemostatic effectiveness data across varying atmospheric pressure conditions. The AI system may include a neural network or decision tree algorithm that processes inputs including ambient atmospheric pressure, target limb circumference, user physiological parameters, and historical effectiveness data to determine optimal initial inflation pressure. The AI model may be trained on datasets correlating atmospheric pressure variations with required tourniquet pressures to achieve consistent arterial occlusion across altitude ranges from sea level to 40,000 feet elevation. In some embodiments, the algorithm may be trained using synthetic datasets for use in space travel and/or other extraterrestrial use (e.g., use on other planets).

The AI-controlled pressure adjustment system may continuously monitor atmospheric pressure changes and automatically modify tourniquet pressure to compensate for environmental variations. The system may sample atmospheric pressure at intervals of 1-10 seconds and detect pressure changes as small as 0.5 psia that could affect tourniquet effectiveness. When atmospheric pressure decreases (such as during aircraft ascent or mountain climbing), the AI system may automatically increase bladder pressure by 5-20 mmHg to maintain consistent compression against reduced external pressure. Conversely, when atmospheric pressure increases (such as during descent or underwater operations), the system may reduce bladder pressure to prevent over-compression and tissue damage.

The artificial intelligence algorithms may incorporate predictive modeling that anticipates pressure adjustments based on rate of atmospheric pressure change and environmental context. The AI system may analyze the rate of pressure change to distinguish between rapid changes (aircraft ascent/descent) and gradual changes (weather variations) and apply different adjustment protocols accordingly. For rapid pressure changes exceeding 1 psia per minute, the system may implement immediate pressure corrections with safety limits to prevent sudden pressure spikes. For gradual changes, the system may apply incremental adjustments over longer time periods to maintain user comfort while preserving hemostatic effectiveness.

The intelligent control system may include safety override mechanisms and manual intervention capabilities to ensure reliable operation across all environmental conditions. The AI system may include fail-safe algorithms that default to conservative pressure settings if sensor readings are inconsistent or if atmospheric conditions exceed the trained parameter ranges. Manual override controls may allow operators to disable automatic adjustments or set fixed pressure targets when AI control is not desired. The system may include audible and visual alerts when atmospheric pressure changes exceed predetermined thresholds or when automatic adjustments reach maximum compensation limits, indicating the need for manual intervention or device reconfiguration.

Machine learning capabilities may enable the AI system to improve performance through operational experience and user feedback. The system may log atmospheric conditions, applied pressures, and hemostatic outcomes to refine the predictive algorithms over time. User feedback regarding tourniquet effectiveness and user comfort may be incorporated into the learning model to optimize pressure adjustment protocols for specific environmental conditions and user populations. The AI system may include wireless connectivity capabilities that allow firmware updates and algorithm improvements based on aggregated performance data from multiple devices deployed across different environmental conditions.

In some embodiments, the system may include temperature compensation mechanisms that account for gas expansion and contraction effects to maintain consistent inflation pressures across varying thermal environments. An integrated temperature sensor may monitor ambient temperature conditions and gas canister temperature, with the sensor comprising a thermistor, thermocouple, or resistance temperature detector (RTD) providing accuracy of ±1° C. over a range from −40° C. to +85° C. The temperature sensor may be positioned within the gas canister assembly or in thermal contact with the gas delivery system to monitor the temperature of the pressurized gas supply.

Temperature-induced pressure variations may be compensated through algorithmic control of gas delivery volume and timing based on ideal gas law calculations. As ambient temperature decreases, the compressed gas in the canister may experience pressure reduction according to Gay-Lussac's law, potentially reducing available inflation pressure by 10-30% in extreme cold conditions. The control system may automatically increase gas delivery volume or extend inflation duration to compensate for reduced gas pressure, ensuring that target bladder pressures are achieved despite temperature-related pressure losses. Conversely, in high-temperature environments where gas pressure increases, the system may reduce delivery volume or implement pressure limiting to prevent over-inflation.

The artificial intelligence system may incorporate thermal modeling algorithms that predict temperature-related pressure changes and proactively adjust inflation parameters. The AI system may process temperature trend data to anticipate pressure variations before they occur, enabling preemptive adjustments to gas delivery timing and volume. The thermal model may account for heat transfer between the gas canister, ambient environment, and user body temperature, predicting equilibrium pressures under different thermal conditions. Machine learning algorithms may refine thermal compensation based on observed performance across varying temperature conditions, improving accuracy of pressure predictions over time.

Multi-zone temperature monitoring may enable compensation for differential thermal effects across system components. The system may include separate temperature sensors for the gas canister, delivery tubing, and bladder assembly to account for thermal gradients that can develop in extreme environments. For example, in cold weather operations, the gas canister may be at ambient temperature while the bladder assembly warms to body temperature, creating pressure differentials that require compensation. The control system may apply different correction factors based on component-specific temperature readings to maintain overall system pressure accuracy.

Thermal stabilization features may minimize temperature-induced pressure variations through insulation and thermal management. The gas canister may include thermal insulation or heat exchange elements that reduce temperature sensitivity of the pressurized gas supply. Phase change materials or thermal mass elements may be integrated into the canister assembly to buffer against rapid temperature changes. The bladder assembly may include temperature-stable materials that maintain consistent pressure-volume relationships across the operating temperature range, reducing the magnitude of thermal compensation required.

The temperature compensation system may include safety limits and thermal protection mechanisms to prevent operation outside safe temperature ranges. The control system may include temperature thresholds that trigger warnings or disable automatic operation when ambient conditions exceed safe operating limits for user safety or device reliability. Thermal protection may include automatic pressure relief when gas expansion due to high temperatures could create dangerous over-pressurization conditions. The system may provide visual or audible alerts when temperature conditions require manual intervention or when thermal compensation reaches maximum adjustment limits, ensuring operator awareness of environmental constraints on device performance.

Adaptive pressure titration algorithms may automatically adjust inflation pressure based on real-time hemostatic effectiveness monitoring and user physiological responses. The AI system may integrate with pulse oximetry sensors, Doppler flow detectors, or photoplethysmography sensors positioned distal to the tourniquet to monitor blood flow cessation and tissue perfusion. Machine learning algorithms may analyze sensor data patterns to determine when effective arterial occlusion has been achieved and automatically halt inflation to prevent over-pressurization. The system may continuously monitor distal pulse signals and adjust pressure incrementally to maintain the minimum effective pressure for hemostasis, reducing tissue damage risk while ensuring bleeding control.

Intelligent timing and duration management may optimize tourniquet application periods based on user condition, injury severity, and physiological monitoring data. The AI system may track application duration and provide automated alerts at clinically significant time intervals (30, 60, 90 minutes) while analyzing user vital signs and tissue perfusion data to recommend optimal release timing. In some embodiments, machine learning algorithms may process data such as (but not limited to) user age, injury type, ambient temperature, and/or physiological parameters to predict safe application duration limits and recommend pressure cycling protocols that maintain hemostasis while minimizing ischemic tissue damage. The system may optionally implement brief automatic pressure reduction cycles when safe to do so, allowing temporary reperfusion to extend safe application time.

Predictive analytics may anticipate pressure loss or device failure before hemostatic effectiveness is compromised. The AI system may monitor pressure trends, gas consumption rates, and mechanical component performance to predict when pressure loss might occur due to gas leakage, bladder fatigue, or mechanical loosening. Machine learning models may identify early warning patterns in pressure decay rates or mechanical stress indicators that precede device failure, providing advance warning to operators. The system may automatically compensate for gradual pressure loss by releasing additional gas or recommend device repositioning when mechanical factors indicate reduced effectiveness.

User-specific optimization algorithms may customize pressure application protocols based on individual physiological characteristics and medical history data. The AI system may incorporate user data including age, blood pressure, cardiovascular condition, and medication history to optimize pressure application protocols for individual users. Machine learning models may adjust pressure targets based on estimated systolic pressure, arterial compliance, and tissue characteristics to achieve effective hemostasis with minimum applied pressure. The system may account for conditions such as hypertension, atherosclerosis, or anticoagulant therapy that affect optimal tourniquet pressure requirements.

Multi-sensor fusion and decision support may integrate diverse physiological and environmental data streams to provide comprehensive tourniquet management recommendations. The AI system may process inputs from pressure sensors, temperature monitors, accelerometers detecting user movement, heart rate monitors, and environmental sensors to provide holistic tourniquet management. Machine learning algorithms may identify complex patterns across multiple data streams that indicate optimal pressure adjustments, application timing, or safety concerns that would not be apparent from individual sensor readings. The system may provide decision support recommendations to medical personnel, highlighting critical changes in user condition or device performance that require immediate attention.

The disclosed system may achieve reliable hemostatic control while maintaining critical safety margins above systolic pressure. Effective tourniquet application may require pressures that exceed systolic blood pressure by at least 30 mmHg to ensure arterial compression despite pressure variations from user movement, tissue swelling, and physiological changes. Conventional tourniquets may apply excessive pressures (300-400 mmHg) to ensure effectiveness, creating unnecessarily large safety margins that increase tissue damage risk. The disclosed system may operate at 200-250 mmHg, providing optimal safety margins of 30-50 mmHg above typical systolic pressures (120-200 mmHg) while maintaining consistent hemostatic effectiveness.

The spring steel constraint mechanism may provide automatic pressure regulation that maintains safety margins during physiological variations. User blood pressure may fluctuate by 20-40 mmHg due to pain, stress, blood loss, and medical interventions. Conventional rigid tourniquets may lose effectiveness when systolic pressure increases or may cause excessive tissue damage when pressure decreases. The disclosed spring-loaded system may automatically adjust applied pressure in response to tissue volume changes, maintaining consistent compression ratios relative to underlying blood pressure. This automatic regulation may ensure that safety margins remain within the optimal 30-50 mmHg range regardless of physiological variations.

The controlled pressure application may enable extended safe application times not achievable with conventional high-pressure systems. Tissue tolerance to tourniquet pressure may be inversely related to applied pressure and application duration. Conventional tourniquets applying 300+ mmHg may cause irreversible tissue damage within 60-90 minutes. The disclosed system operating at 200-250 mmHg may extend safe application times to 2-4 hours while maintaining hemostatic effectiveness. This extended safe operating window may be critical for prolonged field care and/or extended transport times where tourniquet removal is not feasible.

Pressure distribution analysis may demonstrate superior tissue preservation compared to point-contact conventional designs. The distributed pressure application over 3-6 inch contact areas may reduce peak tissue pressures by 40-60% compared to conventional 1-2 inch contact widths. This pressure distribution may significantly reduce the incidence of pressure-related complications including nerve palsy, compartment syndrome, and tissue necrosis. The combination of reduced peak pressures and maintained hemostatic effectiveness may represent a fundamental advancement in tourniquet safety profiles.

The slap band mechanism may eliminate the need for separate positioning and constraint components required in prior art pneumatic systems. Conventional pneumatic tourniquets may require multiple discrete elements: positioning straps, external stiffening bands, pressure distribution pads, and securing mechanisms. Each additional component may increase application complexity, failure points, and training requirements. The disclosed spring steel encircling member may integrate all these functions into a single component that automatically positions, constrains, and distributes pressure upon deployment. This functional integration may reduce the total component count by 60-80% compared to conventional pneumatic tourniquet systems.

The integrated design may provide superior mechanical reliability through reduced interface complexity. Each interface between separate components in conventional designs may represent a potential failure point where pressure can be lost, positioning can shift, or mechanical failure can occur. The disclosed design may minimize these interfaces by integrating positioning, constraint, and pressure distribution functions into the spring steel member itself. The reduced interface complexity may improve system reliability while simplifying manufacturing, quality control, and field maintenance requirements.

The elimination of external stiffening elements may enable conformal pressure application impossible with rigid support systems. External rigid supports in conventional designs may create fixed contact geometries that cannot adapt to individual limb variations. The disclosed spring steel member may provide structural support while maintaining conformability to limb contours, creating custom pressure distribution patterns for each application. This adaptive conformability may optimize vascular compression efficiency while minimizing tissue trauma compared to fixed-geometry conventional designs.

The combination enables a previously unachievable operating mode: controlled arterial restriction with maintained safety margins. Prior art pneumatic tourniquets may be designed for complete arterial occlusion, typically requiring pressures of 250-350 mmHg that completely stop all blood flow. The disclosed combination may operate effectively at lower pressures of 190-250 mmHg, providing sufficient hemostatic control while maintaining minimal arterial flow that prevents tissue ischemia. This controlled restriction mode may be impossible with conventional designs because they lack the uniform pressure distribution and structural stability needed to maintain precise pressure levels over time.

The spring-loaded constraint system may provide dynamic pressure regulation that compensates for physiological variations. During muscle contraction, tissue volume may increase by 15-30%, causing conventional tourniquets to either lose effectiveness or create dangerous pressure spikes. The disclosed spring steel mechanism may provide controlled elastic response that maintains consistent vascular compression pressure despite tissue volume changes. This dynamic regulation may allow sustained operation at the optimal pressure range of 200-250 mmHg with automatic compensation for muscle contraction, user movement, and tissue swelling-a capability not achievable with rigid tourniquet designs.

Clinical testing may demonstrate unexpected hemostatic effectiveness at reduced pressures. The uniform pressure distribution created by the slap band constraint may achieve effective arterial compression at pressures 50-100 mmHg lower than required by conventional point-contact tourniquets. This pressure reduction may significantly decrease the risk of nerve damage, compartment syndrome, and tissue necrosis while maintaining equivalent hemostatic control. The combination may enable safe tourniquet application for extended periods (2-4 hours) that would cause tissue damage with conventional high-pressure designs.

The structural integration between the slap band and pneumatic system eliminates design compromises present in prior art approaches. Conventional pneumatic tourniquets may require external rigid supports or stiffening elements to prevent radial expansion of the inflatable bladder away from the limb. These external supports may add bulk, complexity, and application time while creating pressure concentration points. The disclosed spring steel encircling member may serve dual functions as both the rapid deployment mechanism and the structural constraint system. During inflation, the spring steel member may maintain its coiled configuration under pneumatic pressure, creating an internal skeletal framework that channels all inflation force inward toward vascular compression. This integrated approach may eliminate the need for separate stiffening components while providing superior pressure distribution compared to externally supported designs.

The spring steel constraint mechanism may provide precise pressure vector control during inflation. Unlike rigid external supports that create fixed pressure points, the spring steel member may conform to irregular limb geometries while maintaining consistent circumferential tension. This conforming constraint may ensure that pneumatic pressure is applied uniformly around the limb circumference, eliminating the pressure gradients and dead zones common in conventional designs. The spring steel's elastic properties may allow controlled deformation under extreme pressures while maintaining structural integrity, providing a safety mechanism against over-pressurization that rigid supports cannot offer.

There are many example scenarios where an emergency tourniquet may be necessary or beneficial for application prior to transport to a hospital. The following are example scenarios that may benefit from application of the tourniquet device described herein.

A soldier may sustain a gunshot wound to the thigh with arterial hemorrhage during combat operations. The soldier's dominant arm may be uninjured while the wounded leg may be rapidly losing blood. Using conventional tourniquets, the soldier may struggle to position and tighten a windlass device with one hand while under fire and experiencing pain and blood loss. The disclosed device may enable the following sequence. The soldier may grasp the slap band segment with the uninjured hand and may strike it against the thigh proximal to the wound. The spring steel member may automatically coil around the thigh in less than one second. The soldier may then wrap the remaining band segment and may engage the latch using the pull tab. The soldier may pull the activation pin or lever, causing the gas cartridge to puncture and the air bladder to inflate. The entire process may be completed in under 10 seconds using only one hand. The consistent pressure application may ensure effective hemostasis regardless of the soldier's physical condition or training level.

Multiple victims may sustain traumatic injuries in a vehicle accident, terrorist attack, or other mass-casualty event. First responders may face numerous casualties requiring simultaneous hemorrhage control. Conventional tourniquets may require 1 to 2 minutes per application by trained personnel. The disclosed device may enable a single responder to apply tourniquets to multiple victims in rapid succession. The responder may position the slap band against a victim's limb, may secure the fastening mechanism, and may activate inflation. The responder may then immediately move to the next victim while the first tourniquet maintains pressure. The rapid application time may allow treatment of more casualties within the critical first minutes following injury. Untrained bystanders may also apply the devices effectively due to the intuitive operation, multiplying the available treatment capacity.

A hiker may sustain a severe laceration to the arm from a fall in a remote wilderness area. The hiker may be alone and may be hours from medical assistance. The injured arm may be unable to assist in tourniquet application. Using the disclosed device, the hiker may apply the tourniquet to the injured arm using only the uninjured arm. The slap band mechanism may eliminate the need to wrap the band around the injured limb manually. The pneumatic inflation may provide adequate pressure without requiring the hiker to manually tighten a windlass while experiencing pain and blood loss. The controlled release valve may allow the hiker to periodically reduce pressure during the extended time until rescue, reducing ischemic injury risk while maintaining hemostasis.

A child may sustain a severe laceration requiring tourniquet application. Pediatric limbs may have smaller diameters and may require lower occlusive pressures than adult limbs. Conventional tourniquets may be difficult to size appropriately and may apply excessive pressure. The disclosed device may be provided in multiple sizes with appropriately sized gas cartridges. A pediatric version may use a smaller cartridge providing lower total pressure while maintaining the same rapid application mechanism. The slap band may be sized for pediatric limb circumferences. The distributed pressure from the air bladder may be particularly beneficial in pediatric applications where tissue is more susceptible to pressure injury.

An elderly user with fragile skin and reduced tissue perfusion may require tourniquet application following a fall with arterial bleeding. Conventional tourniquets may cause skin tears and may create excessive tissue trauma in geriatric users. The disclosed device's distributed pressure application may reduce the risk of skin injury. The controlled inflation rate may be adjusted using the valve to provide slower pressure application, allowing tissue accommodation.

A worker may sustain a severe laceration from machinery in an industrial setting. Coworkers without medical training may be the first responders. The high-stress environment and lack of training may make proper conventional tourniquet application difficult. The disclosed device may be stored in industrial first aid stations with minimal instructions required. A coworker may apply the device by following simple steps indicated on the device itself. The slap band application may be intuitive, requiring only striking the band against the limb. The inflation activation may be clearly marked. The consistent pressure application may ensure effective treatment despite the lack of medical training.

A police officer may sustain a gunshot wound to the leg during an armed confrontation. The officer may need to maintain weapon control with one hand while applying a tourniquet with the other hand. The officer may be in an active threat environment requiring rapid treatment and continued defensive capability. The disclosed device may enable one-handed application in under 10 seconds, allowing the officer to maintain weapon control throughout the process. The officer may apply the device while moving to cover. The reliable hemostasis may allow the officer to continue functioning until backup arrives and the scene is secured.

A crew member on a vessel may sustain a severe laceration during rough seas. The vessel may be days from port and medical facilities. The motion of the vessel may make conventional tourniquet application difficult. The disclosed device may be applied quickly despite vessel motion. The slap band may secure to the limb even if the user and person to whom the tourniquet is applied are both moving. The pneumatic inflation may provide consistent pressure regardless of the challenging environment. The controlled release capability may allow pressure management during the extended time until the vessel reaches port, reducing ischemic injury risk while maintaining hemostasis during the multi-day transit.

A construction worker may sustain a severe laceration from a power tool in a remote construction site. The worker may be alone or with coworkers who lack medical training. The injury may involve arterial bleeding requiring immediate intervention. Conventional tourniquets may require two-handed operation that may be difficult for the injured worker to perform alone. The disclosed device may enable the worker to self-apply the tourniquet using only the uninjured hand. The worker may position the slap band segment against the injured limb proximal to the wound. The worker may strike the band against the limb to initiate the coiling action. The spring steel member may automatically wrap around the limb. The worker may then secure the fastening mechanism using the pull tab. The worker may activate the inflation system by pulling the activation element. The entire application process may be completed in under 10 seconds without assistance. The consistent pressure delivery may ensure effective hemostasis despite the worker's lack of medical training. The worker may maintain the tourniquet in place until emergency medical services arrive at the remote location.

A person with a bleeding disorder may require tourniquet application. The person may have hemophilia or may be taking anticoagulant medication that increases bleeding risk. Conventional tourniquets may apply excessive pressure that may cause tissue trauma and may exacerbate bleeding upon removal. The disclosed device may provide controlled pressure application that may minimize tissue trauma. user (e.g., the person or another assisting the person) may select an appropriately sized device for the person's limb. The user may position the band around the limb proximal to the site of the bleeding (e.g., a laceration). The user may activate the inflation system to achieve occlusive pressure. The predetermined pressure delivery may ensure adequate hemostasis without excessive compression. The valve system may allow the user to reduce pressure gradually during the procedure if the person's coagulation status improves. The controlled deflation capability may be particularly beneficial for persons with bleeding disorders. The gradual pressure reduction may allow staged reperfusion that may reduce the risk of rebound bleeding. The user's coagulation parameters may be monitored during the controlled deflation process.

A person may sustain a traumatic amputation requiring immediate hemorrhage control. The person may have lost at least a portion of a limb due to machinery accident, explosion, or other traumatic event. Conventional tourniquets may be difficult to position on a partially amputated limb with irregular anatomy. The disclosed device may provide rapid application despite challenging anatomy. The responder may position the slap band segment around the remaining portion of the limb proximal to the amputation site. The spring steel member may conform to the irregular limb contour during coiling. The responder may secure the fastening mechanism despite the altered anatomy. The responder may activate the inflation system to achieve occlusive pressure. The air bladder may conform to the irregular limb shape during inflation. The distributed pressure may ensure effective vascular compression despite the anatomical disruption. The rapid application time may be critical in traumatic amputation scenarios where blood loss rates may exceed 1000 milliliters per minute. The device may maintain hemostasis during transport to a trauma center for definitive surgical management.

A person may require tourniquet application in a contaminated environment. The person may sustain an injury in a field environment with exposure to dirt, debris, or biological contaminants. Conventional tourniquets with complex mechanical components may be susceptible to contamination that may interfere with function. The disclosed device may provide reliable operation despite environmental contamination. The sealed gas cartridge may be protected from contamination. The pneumatic inflation system may function reliably despite exposure to dirt or moisture. The band materials may be selected for durability in harsh environments. The fastening mechanism may maintain secure attachment despite contamination. The device may be applied quickly to minimize exposure time in the contaminated environment. The external surfaces may be designed for easy decontamination following application. The device may maintain function during transport from the contaminated field environment to a clean medical facility.

A person may require bilateral tourniquet application for injuries to multiple limbs. The person may sustain traumatic injuries to both arms or both legs in a motor vehicle accident or explosion. Conventional tourniquets may require sequential application that may delay hemostasis on the second limb. The disclosed device may enable rapid sequential application to multiple limbs. A single responder may apply the first device to one limb using the rapid application sequence. The responder may immediately apply a second device to the other injured limb. The total application time for both devices may be under 20 seconds. The consistent pressure delivery may ensure effective hemostasis on both limbs without requiring strength-dependent tightening. The responder may monitor both devices during transport. The valve systems on both devices may allow independent pressure management for each limb. The ability to rapidly apply multiple devices may be particularly valuable in mass casualty incidents where multiple persons may require substantially simultaneous treatment.

A person may require tourniquet application during air medical transport. The person may be transported by helicopter or fixed-wing aircraft to a trauma center, for example as part of a medical evacuation (medevac) operation. Conventional tourniquets may require periodic retightening during transport due to vibration and movement of the person. The disclosed device may maintain consistent pressure during air transport without requiring adjustment. The pneumatic inflation system may maintain pressure despite aircraft vibration. The sealed gas system may function reliably despite changes in ambient pressure during flight. The medical crew may monitor the device during transport. The valve system may allow pressure adjustment if needed during the flight. The device may remain in place during transfer from the aircraft to the receiving facility. The consistent pressure maintenance may reduce the workload on the flight crew and may allow them to focus on other aspects of care during transport.

All publications mentioned herein are incorporated herein by reference to disclose and describe the methods and/or materials in connection with which the publications are cited.

### a. Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims, which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the disclosure. Thus, if there are a variety of additional steps that can be performed it is understood that each of these additional steps can be performed with any specific aspect or combination of aspects of the methods of the disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

The tourniquet device in this disclosure may be further referred to as a tourniquet, a tourniquet apparatus, a simple tourniquet, a quick tourniquet, a Tourinquick™, a fast-acting tourniquet, a fast action tourniquet, an instant tourniquet, and the like.

### Tourniquet Devices and Systems

Consistent with embodiments of the present disclosure, a Fast-Acting Tourniquet Device may be provided. This overview is provided to introduce a selection of concepts in a simplified form that are further described below. This overview is not intended to identify key features or essential features of the claimed subject matter. Nor is this overview intended to be used to limit the claimed subject matter's scope. The fast-acting tourniquet device may be used by individuals or medical professionals to control blood flow in medical emergencies, such as those associated with combat wounds and traumatic injuries.

In various aspects, tourniquets, such as those disclosed herein, may be a constricting or compressing device used to control arterial and/or venous blood flow to a portion of an extremity or limb for a period of time. Pressure may be applied circumferentially around a portion of a limb at a desired location. This pressure may be transferred to the walls of blood vessels, causing them to become temporarily occluded or restricted. In further aspects, the disclosed tourniquets may be configured to occlude arterial blood flow following exsanguination to produce a relatively bloodless operative field, preserve limbs, and to minimize blood loss. In emergency settings, the disclosed tourniquets may be used to stop traumatic bleeding such that medical care may be provided in time before an injured person bleeds out. In rehabilitation settings, the disclosed tourniquets may be used to restrict arterial blood flow at a consistent and/or safe pressure for short periods of time during low intensity exercise to more rapidly increase muscle size and strength.

In some instances, a primitive tourniquet may be made from a stick and a rope, leather belt, fabric, or a cloth. The rope may be made into a loop that may fit over the damaged limb and the stick may be inserted through the loop. The loop may be tightened by twisting the stick. This primitive device may stem the flow of blood but side-effects such as soft tissue damage and nerve damage may occur due to the application of unknown, uncontrollable and excessively high pressures and pressure gradients. The present disclosure provides an improved tourniquet device.

In one aspect, the genesis of the present disclosure comes from solving challenges presented by traumatic injuries in military combat situations. In such situations, applications of a tourniquet can save a life. Challenges surrounding the use of a tourniquet may include but is not limited to: improper application of a tourniquet; the amount of training it takes to learn how to properly apply a tourniquet; the time it may take for proper application; and other complexities. Such traumatic injuries are no longer just limited to combat situations. Traumatic injuries may also be part the day-to-day lives as civilians regarding incidents including but not limited to car accidents, work accidents, knifings, shootings, terrorist attacks, and other traumatic injuries.

The present disclosure therefore provides an improved tourniquet device having the ability to save lives. As most civilians lack the training and knowledge to apply a tourniquet, the present disclosure provides a wholly new and innovative tourniquet configured to allow for an extremely simple application of the disclosed tourniquet devices and apparatuses. Such simple application may be utilized by the common person to save a life. The tourniquet device and apparatus may be configured such that no previous knowledge or training may be required. Furthermore, the tourniquet device and apparatus may be configured such that it may be effective, and can be applied within seconds. The present disclosure may be configured such that it may allow a civilian to apply a tourniquet with special forces precision.

In further aspects, the present disclosure may provide a tourniquet device used to stop excessive bleeding from any limb in order to save a life. It may be used in cases of traumatic injuries like car accidents, terrorist attacks, bombings, shootings, knifings, and many other forms of trauma, including puncture wounds and various other injuries. In most of these cases, the injured actually expires due to blood loss and not by impact of the car, shrapnel, or bullet. By applying extreme pressure on and/or around a wound, the blood will stop flowing. The disclosed tourniquet is different because it can allow for easier application which may be quick to put in place, simple to use, easy enough for anyone to place, and less painful to have on while saving a life in under 10 seconds. In still further aspects, the disclosed tourniquets may be self-administered using a single hand.

Clinical literature suggests that higher tourniquet pressures are associated with higher probabilities of tourniquet-related injuries. Modern pneumatic tourniquet systems may be microcomputer-based, allowing more accurate and automatic pressure control and many important safety features to minimize the risk of user injury. Recent trends toward personalized care has increased user safety by allowing perioperative staff (and/or others applying the tourniquet to a user) to measure and select a personalized tourniquet pressure based on the user's Limb Occlusion Pressure. Moreover, perioperative staff may select and apply a personalized tourniquet cuff that can adapt the shape of the tourniquet cuff to a wide range of non-cylindrical limb shapes.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

According to various aspects, disclosed herein is a device for controlling local blood flow. In further aspects, the device generally comprises a pressurized gas source; a switch or mechanical barrier in operable communication with the pressurized gas source, the switch configured to selectively release or dispense a predetermined volume of gas in a gas stream having a predetermined flow rate and a predetermined flow path; and a band comprising a bendable encircling member and an air compartment or air bladder, the air compartment in operable communication with the gas stream; the encircling member having an extended state and coiled state configured to circumferentially couple around a portion of a user's body; and a fastening means configured to releasably secure the band around the portion of the user's body. In still further aspects, the gas stream may be configured to fill at least a portion of the compartment effective to cause the band to exert a predetermined level of pressure against the portion of the user's body effective to reduce local blood flow. In yet further aspects, the predetermined level of pressure is effective to stop all blood flow to the area.

The pressurized gas source may comprise a compact gas canister containing compressed air or nitrogen at pressures of 800 to 1200 psi, with total gas volumes of 8 to 16 cubic inches. The canister may be sized to provide multiple inflation cycles, with each inflation requiring approximately 0.5 to 2.0 cubic inches of gas depending on limb circumference and desired pressure. The gas canister may include a pressure regulator that reduces the stored pressure to working pressures of 50 to 100 psi before delivery to the air bladder. The regulator may comprise a spring-loaded diaphragm mechanism or a precision needle valve system that maintains consistent output pressure despite varying canister pressure as gas is consumed.

The predetermined flow rate control may be achieved through calibrated orifice sizing and valve timing mechanisms. The first predetermined inflation rate may deliver gas at 0.1 to 0.3 cubic inches per second, achieving full bladder inflation in 10 to 30 seconds for gradual pressure application. The second predetermined inflation rate may deliver gas at 0.5 to 1.5 cubic inches per second, achieving full inflation in 2 to 8 seconds for rapid hemostatic control. The flow rate differential may be controlled by a dual-orifice valve system, where the activating switch selects between a smaller orifice (slow rate) and a larger orifice (fast rate), or opens both orifices simultaneously for maximum flow.

The air valve toggle mechanism may comprise a rotary selector valve or a multi-position sliding valve that provides positive selection between inflation rates. The valve may include detent positions that provide tactile feedback to confirm rate selection. In one embodiment, the valve may be a three-position rotary valve with positions for “slow inflation,” “fast inflation,” and “off/vent.” The valve body may contain precision-machined orifices of different diameters, such as 0.020 inches for slow inflation and 0.060 inches for fast inflation. The valve may include internal sealing elements such as O-rings or valve seats that prevent gas leakage between positions.

The pressure control system may achieve the specified 190-250 mmHg operating range through bladder volume and constraint design optimization. The air bladder may have an internal volume of 2 to 8 cubic inches when fully inflated, with bladder dimensions of approximately 6 to 12 inches in length and 2 to 4 inches in width. The bladder material may be flexible polymer such as thermoplastic polyurethane (TPU) with a thickness of 0.005 to 0.015 inches, providing sufficient strength to contain the required pressures while maintaining flexibility for limb conformance. The spring steel constraint mechanism may limit bladder expansion to create the desired pressure-to-volume relationship, where full gas delivery results in bladder pressures of 200 to 250 mmHg against typical limb resistance.

In further aspects, the encircling member (or “slap band member”) may comprise a restorably deformable member having a one-way and/or directional potential energy driven state transition. In still further aspects, the encircling member can comprise a spring mechanism or means for producing a coiling action, such as a spring steel. In yet further aspects, the band may comprise a plurality of different segments or portions. The encircling member may be disposed within a first portion or segment of the band. For example, the band may comprise an assistive segment containing the encircling member or spring mechanism and a non-assistive wrapping segment without an encircling member. The band may comprise an assistive segment containing the encircling member to assist with initial clamping or encircling of the band around the body. In further aspects, after the assistive segment is slapped, clamped or otherwise secured against the body, another segment can then be wrapped around the body part and secured to the outside surface of the band using a fastener or other fastening means.

The single-handed application procedure may be accomplished through a specific sequence of operations designed for rapid deployment under emergency conditions. The operator may grasp the band assembly with the dominant hand, positioning the spring steel end away from the fastening mechanism. The band may be struck against the target limb with sufficient force to trigger the spring steel deployment, typically requiring 2 to 8 pounds of impact force applied over 0.1 to 0.3 seconds. The spring steel member may automatically wrap around the limb in less than 0.5 seconds, providing initial positioning and constraint.

Following automatic deployment, the operator may secure the fastening mechanism using the same hand that applied the device. The fastening system may be designed for intuitive operation, with the hook-and-loop strips or buckle mechanism positioned for easy access. The fastening operation may require 3 to 10 seconds to complete, with tactile feedback confirming secure engagement. The total single-handed application time from initial contact to secured positioning may be 5 to 15 seconds, significantly faster than conventional two-handed tourniquet application procedures.

The precise pressure control during single-handed operation may be achieved through pre-calibrated gas delivery systems and visual pressure indicators. The gas canister and valve system may be pre-set to deliver the appropriate gas volume for target pressure achievement, eliminating the need for pressure adjustment during emergency application. A pressure gauge or indicator may provide immediate visual confirmation that effective pressure has been achieved. The gauge may include color-coded zones indicating insufficient pressure (red, below 180 mmHg), effective pressure (green, 180-280 mmHg), and excessive pressure (red, above 280 mmHg).

The gradual deflation system may provide controlled pressure reduction for safe device removal and restoration of blood flow. The deflation mechanism may include a precision needle valve or adjustable orifice that allows gas release at controlled rates of 0.05 to 0.2 cubic inches per second. This controlled deflation may reduce bladder pressure by 20 to 50 mmHg per minute, allowing gradual restoration of circulation while monitoring for bleeding recurrence. The deflation system may include detent positions or flow rate markings that provide consistent deflation rates regardless of operator technique.

Operational safety protocols may include timing guidelines and physiological monitoring requirements to prevent complications from extended application. The device may include timing indicators or audible alarms that alert operators to application duration milestones at 30, 60, and 90 minutes. Pressure monitoring may be performed at 5 to 15 minute intervals to ensure maintained effectiveness and detect pressure loss that could indicate device failure or improper application. The safety margin maintenance may be verified through periodic blood pressure measurement, ensuring that tourniquet pressure remains 30 to 50 mmHg above systolic pressure throughout the application period.

The spring steel encircling member may comprise a flat spring steel strip having specific dimensional and material characteristics that enable the one-way directional potential energy driven state transition. The spring steel may have a thickness of approximately 0.010 to 0.030 inches, a width of 0.5 to 2.0 inches, and a length of 8 to 16 inches, depending on the intended limb circumference. The spring steel may be manufactured from high-carbon steel alloys such as 1074, 1084, or 1095 carbon steel, heat-treated to achieve a hardness of 45-50 HRC (Rockwell C scale). The spring steel may be pre-stressed during manufacturing to create a natural coiled configuration with a resting diameter of 2 to 6 inches.

The one-way directional energy release mechanism may function through controlled elastic deformation and energy storage principles. In the extended state, the spring steel member may be held in a substantially straight configuration against its natural coiled tendency, storing potential energy in the form of elastic strain. The spring steel may be constrained in this extended state by the fabric layers of the band during storage and handling. When the band is struck against a limb surface, the impact force may overcome the fabric constraint, allowing the stored elastic energy to drive the spring steel member rapidly from the extended state to the coiled state. The transition may occur in less than 0.5 seconds, with the spring steel member wrapping around the limb in a helical pattern.

The coiling action may be achieved through specific geometric and mechanical design parameters. The spring steel member may be pre-formed with a slight helical twist along its length, creating a natural tendency to wrap in a spiral pattern rather than forming a simple circle. The twist angle may be approximately 15 to 45 degrees over the length of the member. The spring steel may be positioned within the band assembly such that one end is anchored near the fastening mechanism while the free end extends toward the opposite end of the band. During coiling, the anchored end may serve as a pivot point while the free end sweeps around the limb circumference, creating consistent contact pressure distribution.

In various aspects, the encircling member is generally configured to encircle around a limb upon striking or slapping it against the limb or body part. For example, the encircling member is configured to encircle around a limb upon striking or slapping an area of the band corresponding to the location of the member against the limb. In further aspects, the encircling member may be configured to encircle around a limb upon striking or slapping it against the limb effective to cause it to go from an erect or extended state to a coiled state. In some aspects, the encircling member and/or assistive segment may comprise a thumb tab or other protrusion for guiding and/or assisting with an initial securing step.

In various aspects, the device may comprise one or more housings or casings for housing one or more device components, for example, the pressurized gas source. In further aspects, the air compartment of the band may comprise an air bladder or air chamber, or the like. The device may also include at least one gas or air supply line configured to contain and/or carry the gas stream. The gas supply line may be in fluid communication with the pressurized gas source, and/or with the air compartment, for example, to carry the gas from gas source into the air compartment for inflation. The pressurized gas source generally comprises at least one of a pressurized gas vessel, bottle, canister, cylinder, tank, and cartridge, and the like. In further aspects, the pressurized gas source may be contained within a housing, and may be detachably connected to the band.

In further aspects, the device may comprise a housing or casing for housing one or more device components. In still further aspects, the housing or casing may be configured for containing the pressurized gas source and/or container. In yet further aspects, the housing can be any shape, and preferably conforms to most hands, particularly fingers. For example, various areas may be concave or convex so to conform to areas of the body to which the device may be held. The housing can be any shape, and preferably is in the shape of a three-dimensional polygon and the housing walls define an interior space or interior sections for containing the operating elements of the invention. Any other shape (as used herein, the term shape is used in the broad sense of three-dimensional works) may be employed, so long as the shape is large enough and structured so as to be able to contain the various working components of the invention as more fully disclosed herein. The housing may be a generally hollow structure sized to contain a pressurized gas source and/or container. In yet further aspects, the device components can be all housed within the housing such that they are protected from, for example, for inadvertent shutting off or the environmental elements. In even further aspects, the housing can be comprised of plastic, plastic composite, reinforced plastic, metal, metal composite, or combinations thereof. In still further aspects, the plastic can comprise polypropylene, impact resistant plastic, or the like. The device may comprise a casing, such as band, strap, or wrap, for containing or connecting the air bladder as disclosed herein.

In further aspects, the pressurized gas source may comprise an air-tight chamber comprising a compressed gas cartridge and cartridge puncture element for releasing the pressurized gas from the gas cartridge. The chamber may be configured to contain the gas cartridge, puncture element and gas released from the gas cartridge. In still further aspects, the chamber may comprise a spring element or other means for exerting a force against an end of the gas cartridge and/or puncture element effective to cause the puncture element to release gas from the gas cartridge. In some aspects, the air chamber may comprise a housing having a plurality of portions for containing the puncture element, gas cartridge, and/or spring element.

In various aspects, the chamber may comprise removable obstructing means for mechanically obstructing the puncture element from puncturing the gas cartridge to release gas. In further aspects, the puncture element may be configured to puncture the gas cartridge to release gas upon removal of the obstructing means. To this end, the puncture element cooperates with the spring element to puncture the gas cartridge to release gas upon removal of the obstructing means. In some aspects, the pressurized gas source may be an external source remotely located from the device. In other aspects, the band may be configured to connect to external pneumatic system or air source to ease the user out of the tourniquet instead of cutting it off directly.

In further aspects, a switch or activating element may be configured to control and activate flow of gas from the pressurized gas source. The switch may be configured to control the flow of gas flowing through a gas supply line in fluid communication with the pressurized gas source. In still further aspects, the switch may be configured to control gas flow by causing a valve position to move from a closed position to an open position. In yet further aspects, the switch may be in operable communication with a puncture element for releasing pressurized gas from the pressurized gas source, for example, the switch may be operable communication with a gas cartridge puncture element for releasing pressurized gas from a gas cartridge. In some aspects, the switch may comprise a removable tab or barrier, and wherein the device may be activated by the removal of the tab or barrier. In other aspects, the switch may be a removable tab or barrier between the gas source and a puncture element for releasing pressurized gas from the gas source. In further aspects, the switch may be a removable tab or barrier between a gas cartridge and a gas cartridge puncture element for releasing pressurized gas from the gas cartridge. In yet further aspects, the switch may comprise an actuating mechanism. In still further aspects, the switch may comprise an actuating mechanism connected to a puncture element, for example, an actuating mechanism hingedly connected to a puncture element. To this end, the switch may be an actuating mechanism hingedly connected to a puncture element and is configured to activate gas release from gas source by pulling on it.

In various further aspects, the switch may comprise a toggle, lever, tab, pin, key, push-button, pull-cord, or the like. The switch may be in operative communication with a valve for selectively causing a valve to move from at least one of: a closed position to an open position or an open position to a closed position to control gas flow from the gas source, for example, a valve of an air cartridge. The switch can be secured to the housing at any convenient position where it may readily be actuated or pulled, and may be mechanically connected in a known manner between the pressurized gas source to control the gas dispensed into a gas stream or air bladder. As described herein, switch can be a pull tab, common on/off switch, such as a toggle, lever, push-button, or other switch. The disclosure herein contemplates any type of switch that would be practical with a pressurized gas source. As shown herein, the switch, may be a pull tab or actuating lever.

In further aspects, switch or regulator may be configured to control a flow rate of gas flowing from the pressurized gas source (or a gas supply line in fluid communication with the pressurized gas source). In still further aspects, the switch may be configured to control gas flow by moving a valve position from a closed position to an open position. In yet further aspects, the switch may comprise an actuating mechanism. In some aspects, the switch comprises a button triggered ball valve. In other aspects, the device may comprise a single valve for dispensing and/or releasing the pressurized gas. In further aspects, the switch may be in operative communication with a valve for selectively moving the valve from at least one of: a closed position to an open position and an open position to a closed position to control gas flow from gas source.

In some aspects, the device may be self-regulating. In other aspects, the device may include a gas regulator or other means for controlling the gas flow rate and/or gas volume from the gas source. In yet other aspects, the device may comprise a flow control component configured to control a gas flow rate and/or volume. The flow control component may be in fluid communication with the gas stream, and configured to control a flow rate and/or volume of a gas stream. In further aspects, the flow control component may be configured to control gas flow rate entrance into the air compartment and/or exit from the air compartment. In some aspects, the flow control component may be in fluid communication with the air compartment, and configured to control an entrance flow rate and/or entrance volume of gas. In further aspects, the flow control component may be in fluid communication with the air compartment and configured to control release of gas from the air compartment. In still further aspects, the flow control component may be in fluid communication with the air compartment, and configured to control an exit flow rate and/or exit volume of gas.

In further aspects, the placement of the pressurized gas source is variable so long as the gas flow from pressurized gas source can be dispensed so as to produce sufficient inflation and pressure for the blood flow control effects as described herein. Pressurized gas source can be placed within housing during manufacture or at any time after manufacture. In further aspects, while the pressurized gas source described herein can be permanently mounted in or on the device housing or device structure, this is not a requirement. For example, the pressurized gas source can be a cartridge configured to be removably mounted in or on a portion of the device, e.g., to allow interchange and/or replacement of the pressurized gas source. Such configurations allow users, first responders, or other medical personnel to select desired pressure profiles, and blood flow reducing-reducing properties or levels in the device, e.g., for customization purposes, for personal preferences, to match desired treatment use, a subject's physical characteristics, a subject's symptoms, or the like. To this end, the housing may use an ingress and egress element, as various embodiments of the pressurized gas source may require exchanging or replacement on occasion.

In various aspects, the pressurized gas source may comprise at least one of a pressurized gas vessel, bottle, cylinder, tank, and cartridge, and the like. In further aspects, the pressurized gas source may be contained with the device housing. In still further aspects, the pressurized gas source may be detachably connected to the device, or otherwise replaceable. In some aspects, the pressurized gas source may comprise an airtight chamber comprising with a compressed gas cartridge and cartridge puncture element for releasing the pressurized gas; wherein the chamber is configured to contain the gas cartridge, puncture element and gas released from the gas cartridge. In other aspects, the pressurized gas source may be an external gas source remotely located from the device. In further aspects, the pressurized gas source may be an external powered air compressor.

In various aspects, the fastening means or fastening component may comprise a hook and loop, hook and catch, latch, buckle, clip, snap, or the like, or any combination thereof. In some aspects, fastening means is a latch, buckle, or the like. In other aspects, the fastening means or component may use a cinching mechanism for securing the band around the portion of the user's body. In further aspects, the fastening means or component may be a hook and loop arrangement. For example, the fastening means or component may be a hook connected a first end of the band and a plurality of loops disposed on an outer surface of the band.

In further aspects, the band may comprise a cuff, wrap or structural means for releasably encircling or wrapping around a portion of a user's body or limb. The band ma y be sized and dimension to releasably encircle any desired body limbs, such as a thigh or arm. To this end, the band may have a closed diameter of from about 1 in to about 36 inches, for example from about 12 inches to about 24 inches. As discussed in more detail below, the band can be comprised of neoprene or other soft material on some portions, and nylon and/or other tough and durable material on other portions.

In still further aspect, the components can be integrally or mechanically attached to other components. In a yet further aspect, the disclosed components can be connected, attached, or mounted using a connecting means, the connecting means comprising a fitting, insert, adhesive, brazing, soldering, welding, spot weld, screw with nut, rivet, fitting, insert, threading, friction fit, or snap-fit or a combination thereof. In further aspects, the device components can be attached using a connecting means. In still further aspects, the connecting means can comprise a fitting, insert, adhesive, brazing, soldering, welding, spot weld, screw with nut, rivet, threading, friction fit, snap-fit, twist-lock, or interlocking mechanism or a combination thereof. In yet further aspects, the connection can be achieved using a snap, friction fitting, snap ring, O-ring, pressure fitting, clip, clasp, and the like. The snap ring or O-ring can be retained within a groove to accommodate the snap ring or O-ring. In a further aspect, the device can comprise an engagement means for coupling and holding components together. In a further aspect, the engagement means can be a screwing mechanism, a click-lock mechanism, or friction mechanism, or the like.

In further aspects, the device may comprise one or more air bladder for inflation: a band using neoprene material that is fabric backed and then stitched to a nylon strip. In other aspects, the present disclosure may comprise a band with slap band portion which may act as a medium of application to the body part. The main body of the band may comprise silicone, plastic, vinyl, nylon, cloth, cotton, or other textured fabric. In yet other aspects, the present disclosure may comprise air chamber such that it may act as a trigger for inflating the air bladder. In yet other aspects, the present disclosure may comprise a valve wherein one or more types of valves may be configured such that they may be directly coupled (e.g., by EMTs) to one or more devices that may control the release of pressure. In even further aspects, a tourniquet device can comprise: an inflatable band comprising a slap band segment with a spring mechanism or member; a latch or fastening means; a pressurized air source and air bladder; and an optional air valve for controlling inflation level of the air bladder.

The present disclosure may provide for civilian and professional uses. For civilian use, the tourniquet may be used in med kits or first aid kits. For professional uses, the tourniquet device may be used by EMS, paramedics, fire fighters, police officers, and for urgent care. Moreover, the tourniquet device may be configured for use within the military. A tourniquet device and systems for using a fast-acting tourniquet device. A fast acting tourniquet device may be provided, comprising: a slap band having a spring mechanism wherein the slap band is the medium of application of the device to a limb member; an air bladder configured to provide an inflation circuit; an air canister combustion chamber configured to trigger activation of the inflation circuit of the air bladder; a latch configured to close the air bladder around the limb member; and an air valve configured to control release of the device.

As briefly described above, the present disclosure relates, in various aspects, to a tourniquet device. In one aspect, a tourniquet device can comprise: a band with slap portion having spring or coil mechanism, or other mechanical means for one-handedly securing the slap portion of the band around a body part by slapping band against said body part; a latch or hook, or other fastening means; an air canister chamber comprising a pressurized and air bladder; and an optional air valve for controlling inflation level and/or release of the band against the body part.

In still further aspects, the slap-band (i.e., spring or coiling) mechanism as described herein may allow for quick attachment/placement or initial securing of the band to the desired portion of the body. In yet further aspects, the user can wrap the remaining portion of the band and can use an attached latch to secure the band having the air bladder/fabric around the limb. In another aspect, the air canister chamber can then operate to inflate the air bladder and constrict local blood flow, i.e. to the limb located proximate to the secured band. In further aspects, the release of the tourniquet device may be controlled by an air valve. The air valve may be configured to allow a slow release of air, therefore not allowing the development of the potentially deadly compartment syndrome. In other aspects, the tourniquet device may include a system to regulate the control of inflation level and/or pressure of tourniquet device.

The pressure monitoring and maintenance system may include integrated pressure sensing and feedback mechanisms to ensure consistent hemostatic effectiveness. The system may incorporate a miniature pressure transducer or strain gauge sensor positioned within the air bladder or connected via a pressure tap. The sensor may provide real-time pressure readings with accuracy of #5 mmHg over the operating range of 150 to 300 mmHg. The sensor output may be displayed on a digital readout or analog gauge visible to the operator, allowing verification that target pressures are achieved and maintained.

The system may include automatic pressure compensation mechanisms that account for gas cooling and bladder stretch over time. As compressed gas cools from ambient temperature to body temperature, pressure may decrease by 10 to 20 mmHg. The bladder material may exhibit stress relaxation that reduces pressure by an additional 5 to 15 mmHg over the first 10 minutes of application. To compensate for these effects, the initial inflation pressure may be set 20 to 35 mmHg above the target pressure, or the system may include a secondary gas reservoir that automatically releases additional gas when pressure drops below a predetermined threshold.

The spring-like response mechanism may provide dynamic pressure compensation through controlled elastic deformation of the spring steel constraint system. During muscle contraction, limb circumference may increase by 10 to 25%, creating tissue volume expansion that would normally cause pressure loss in rigid tourniquet systems. The spring steel encircling member may stretch elastically by 5 to 15% of its circumference, accommodating tissue expansion while maintaining spring tension that preserves contact pressure. The spring constant of the steel member may be calibrated to provide 15 to 40 pounds of restoring force per inch of extension, ensuring that pressure is maintained within +10 mmHg of the target value during normal muscle activity.

The pressure compensation system may include mechanical and pneumatic elements that respond to circumferential changes. As the limb expands, the spring steel member may stretch while the air bladder volume decreases proportionally, creating a pressure increase that compensates for the reduced contact area. The bladder material properties may be selected to provide controlled stretch characteristics, with elastic modulus values that complement the spring steel response. The combined system may maintain contact pressures within 200 to 250 mmHg despite limb circumference variations of up to 20%, providing consistent hemostatic effectiveness during user movement and physiological changes.

Safety mechanisms may include pressure relief valves, maximum pressure limiters, and emergency release systems to prevent over-pressurization and tissue damage. The system may incorporate a pressure relief valve that automatically vents gas when bladder pressure exceeds 300 mmHg, preventing dangerous over-pressurization that could cause compartment syndrome or nerve damage. The relief valve may be a spring-loaded poppet valve or burst disc that opens at predetermined pressure levels with accuracy of +15 mmHg. The valve may vent gas at a controlled rate of 0.1 to 0.5 cubic inches per second, allowing gradual pressure reduction while maintaining hemostatic effectiveness.

The emergency release system may provide rapid deflation capability for immediate pressure relief in case of complications. The system may include a manual deflation valve or emergency release cord that allows complete bladder deflation in 5 to 15 seconds. The emergency release may bypass normal pressure control systems and provide direct venting to atmosphere through a large-diameter port. Visual and tactile indicators may confirm that the emergency release has been activated and that pressure has been relieved.

The spring-loaded diaphragm pressure regulator may comprise a flexible diaphragm element made from reinforced rubber or elastomeric material with a thickness of 0.030 to 0.080 inches. The diaphragm may be positioned between an inlet chamber connected to the high-pressure gas source and an outlet chamber connected to the bladder delivery system. A calibrated spring may apply predetermined force against the diaphragm, with spring constants of 10 to 50 pounds per inch depending on the desired output pressure. As inlet pressure decreases due to gas consumption, the spring force may maintain consistent outlet pressure by allowing the diaphragm to move and maintain the pressure differential across the regulator.

The spring-loaded poppet valve may comprise a valve body with a precision-machined seat and a spring-loaded poppet element that seals against the seat under normal operating conditions. The poppet may be a spherical or conical element made from stainless steel or brass, with sealing surfaces finished to 32 microinch surface roughness. The spring may provide closing force of 5 to 25 pounds, calibrated to open when inlet pressure exceeds 300 mmHg±15 mmHg. The valve body may include adjustment mechanisms such as threaded spring preload adjusters that allow field calibration of the opening pressure.

In yet another aspect, the tourniquet device coupled to the pneumatic system may be configured to ease the user out of the tourniquet instead of cutting it off directly. In further aspects, the tourniquet device may provide components which may be configured as provided in this present disclosure. For example, the slap-band (spring) mechanism, the air canister chamber, and the air bladder may be provided.

In one aspect, the device may comprise a nylon strap, a buckle, a bladder, and an air canister. In a further aspect, the device may comprise a slap side, a latch having a hook and loop, an inflatable bladder having a synching action. In a yet further aspect, the device may comprise a compressed air can having a spring, a housing lower, a housing upper, a tube for airflow, a pull cord, a syringe, and a stopper or spacer. In a still further aspect, the device may comprise a retaining band, a bladder skin, a canister housing, a gas canister (e.g., containing compressed air, carbon dioxide, nitrogen gas, and/or the like), a pull latch, an inflatable bladder, a puncture tool, and a latch synched to a strap down. In an even further aspect, the device may comprise a strap, a buckle and pull cord. In a yet further aspect, the device may comprise an aluminum buckle at the end of tourniquet, a spring steel base, an insert into a slot or pass through loop, and thumb print for context information. In even further aspects, the device may comprise an interior touch point, stitched for latch, a hook, neoprene fabric, and a spring steel touch point.

The fastening mechanism may comprise a hook-and-loop system, buckle mechanism, or snap-fit connector designed to provide secure attachment while enabling rapid release under emergency conditions. In one embodiment, the fastening mechanism may include a hook-and-loop strip extending 2 to 6 inches along the band width, with hook material on one end and loop material on the opposite end. The hook-and-loop materials may provide holding strength of 5 to 15 pounds per square inch, sufficient to maintain band position against pneumatic expansion forces while allowing manual release by pulling with 10 to 25 pounds of force.

Alternative fastening mechanisms may include quick-release buckles or cam-lever systems that provide positive mechanical engagement. A buckle mechanism may comprise a plastic or metal buckle body with a hinged lever that engages teeth or ridges on a strap portion. The buckle may be designed for one-handed operation, allowing the user to engage the fastener by inserting the strap and pressing the lever, and to release by lifting the lever while pulling the strap. The buckle mechanism may provide holding strength of 50 to 200 pounds while requiring only 5 to 15 pounds of release force when the lever is actuated.

The multi-layer band construction may provide structural integration of the spring steel member, air compartment, and fastening systems while maintaining flexibility and comfort. The top layer may comprise a durable outer fabric such as nylon ripstop, polyester canvas, or aramid fiber weave with a thickness of 0.005 to 0.020 inches. The top layer may provide abrasion resistance, puncture protection for the internal components, and attachment points for the fastening mechanism. The middle layer may comprise a flexible separator material such as thin polyurethane film, polyethylene sheet, or fabric membrane that isolates the spring steel member from the air compartment while allowing controlled flexing during deployment.

The bottom layer may comprise a soft, biocompatible material that contacts the user's skin or clothing during application. The bottom layer material may be closed-cell foam, fabric padding, or flexible polymer with a thickness of 0.020 to 0.100 inches. The material may provide cushioning to distribute pressure evenly and prevent pressure points that could cause tissue damage. The bottom layer may include perforations or breathable construction to allow air circulation and moisture management during extended wear periods.

In further aspects, the device may comprise an air cartridge which allows for inflation and snapping into place of the tourniquet device. In still further aspects, the device may comprise an interior touch point, stitched for latch, a hook, neoprene fabric, and a spring steel touch point. In yet further aspects, the device may comprise a retaining band, a bladder skin, a canister housing, a gas canister (e.g., containing compressed air, carbon dioxide, nitrogen gas, and/or the like), a gas cartridge, a key to activate a puncture mechanism which mechanically obstructs spring-loaded puncture tool, a bold, a spring, a pin or puncture mechanism, and a valve assembly. In yet further aspects, the device may comprise a housing A, a housing B, a housing C, a gas cartridge, a spring, a valve system, a pin or key, and a puncture mechanism.

The disclosed tourniquet devices may be advantageous when it comes to both the civilian market and the professional medical market. For example, the civilian market, the present disclosure may provide for an emergency tourniquet device for first aid use. With previous tourniquets, civilians may have lacked the training required to gain the knowledge needed. For example, in the professional market, the present disclosure may provide a tourniquet device that may be quicker and more efficient than the ones currently available.

TABLE 1
Advantages and solutions provided by the present disclosure
compared with existing problems in the prior art.

| | Solutions Provided by the Present |
|---|---|
| Existing Problems In Prior Art | Disclosure |
| A tourniquet requires two hand operation of a | The slap-band spring allows for one-handed |
| tourniquet. | operation of the tourniquet device. |
| A tourniquet takes longer periods of time to put | The tourniquet device is configured to operate |
| in place. | in a short period of time. |
| Proper use of a tourniquet may require specific | The tourniquet device may be configured to |
| levels of training. Even with various levels of | utilized the slap-band spring and air cannister |
| training, applying a tourniquet may take | pull tab to allow for extremely quick application |
| anywhere from 30 seconds to 10 minutes | (For example, sub 7 seconds dominant hand, |
| | sub 10 seconds weak hand) |
| The proper application of a tourniquet may be | The tourniquet device may provide intuitive |
| extremely complicated. Most tourniquets may | directions for use that may be so simple such |
| have one or more pages of directions for proper | that the context clues on the tourniquet may |
| use. | serve as instructions. |
| Application of pressure for a tourniquet may | The tourniquet device may provide consistent |
| depend on the strength of the applier which | pressure such that it may not depend on the |
| allows for human error. Additionally, the | strength of the applier. The present disclosure |
| twisting of a metal rod, plastic piece, or | may be configured such that the constriction of |
| wooden bar with a rope may be used until | blood flow is mechanized (through the air |
| blood flow stops. This also allows for human | canister and bladder) at a pre-designated |
| error and inconsistent application of pressure. | pressure (pound per square inch) range. |
| A tourniquet may continue to be painful to put | The tourniquet device of the present disclosure |
| on/wear. | may be less painful to have on because the |
| | surface area (contact with skin) may be much |
| | larger, therefore the pressure may be spread |
| | out over a greater surface area of space. |

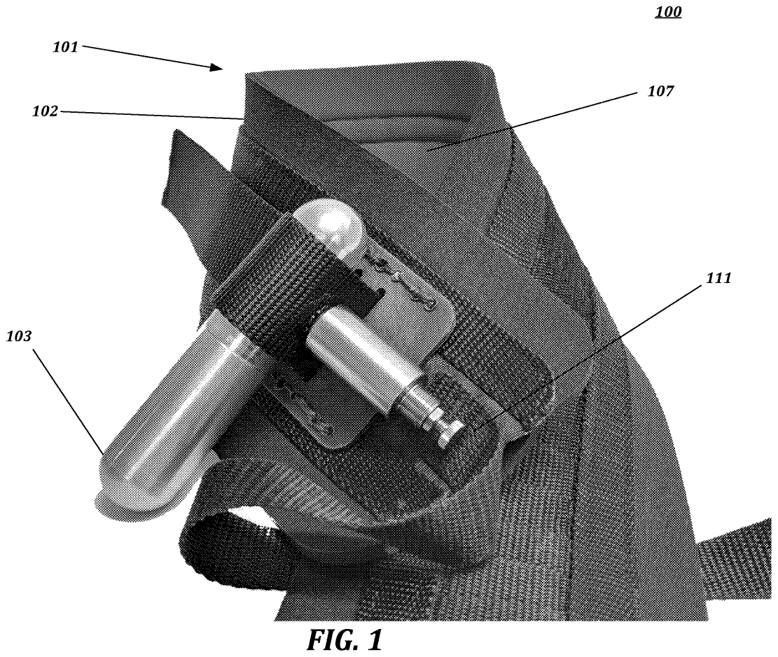

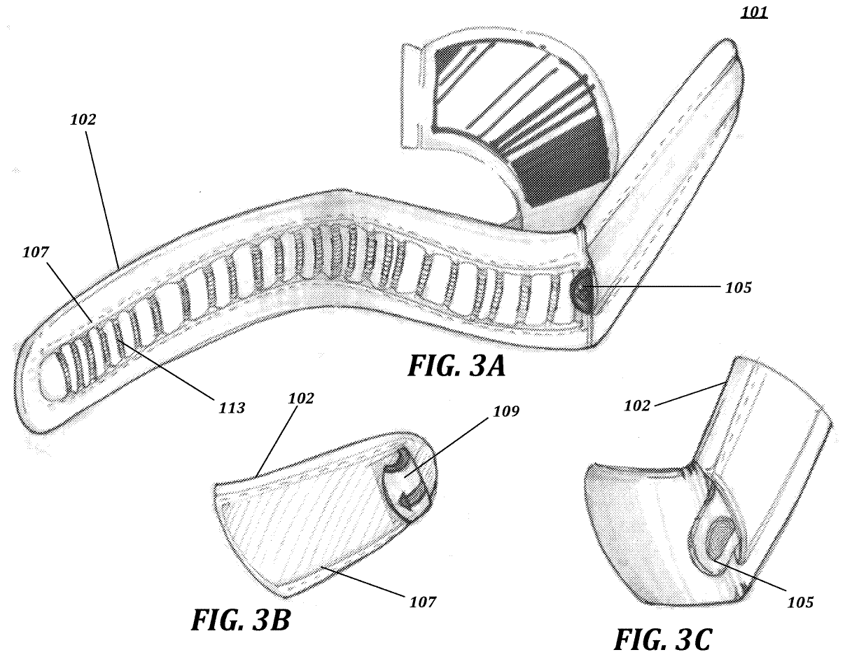

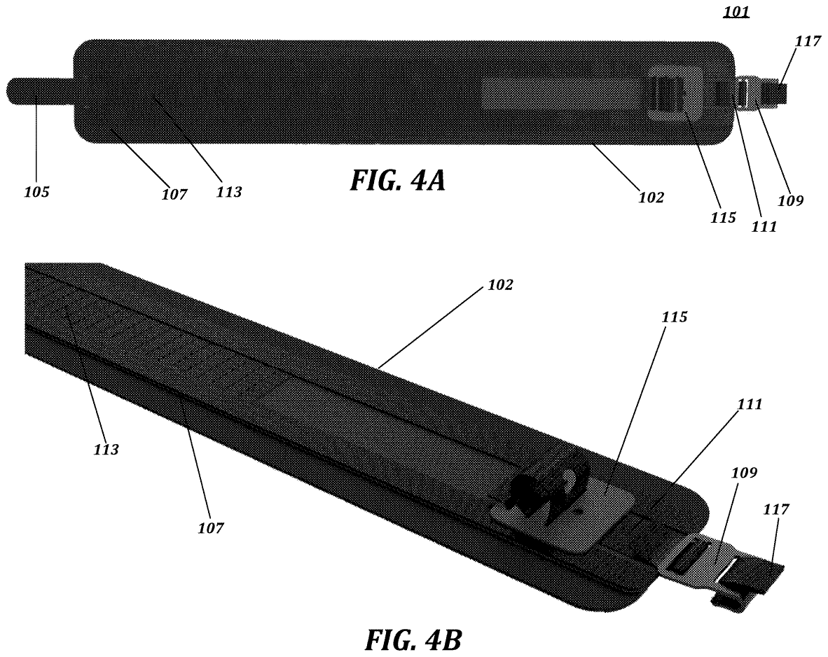

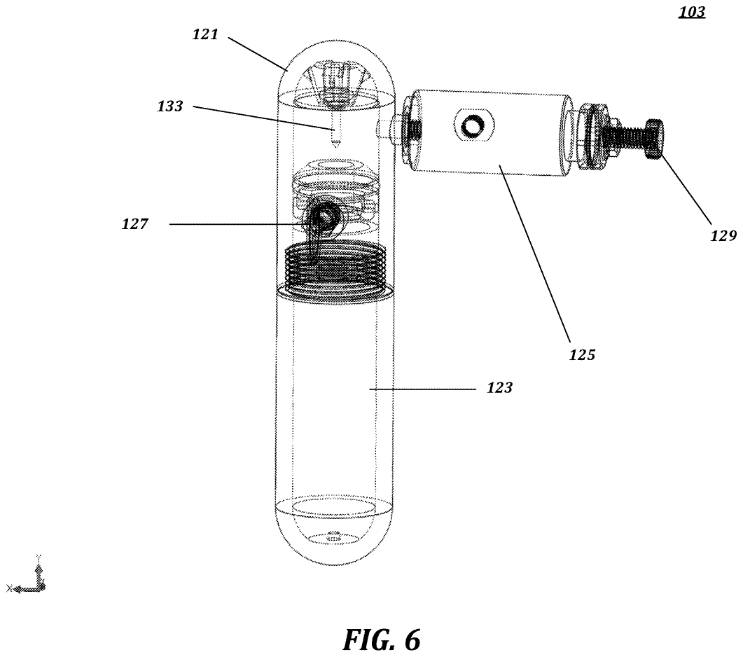

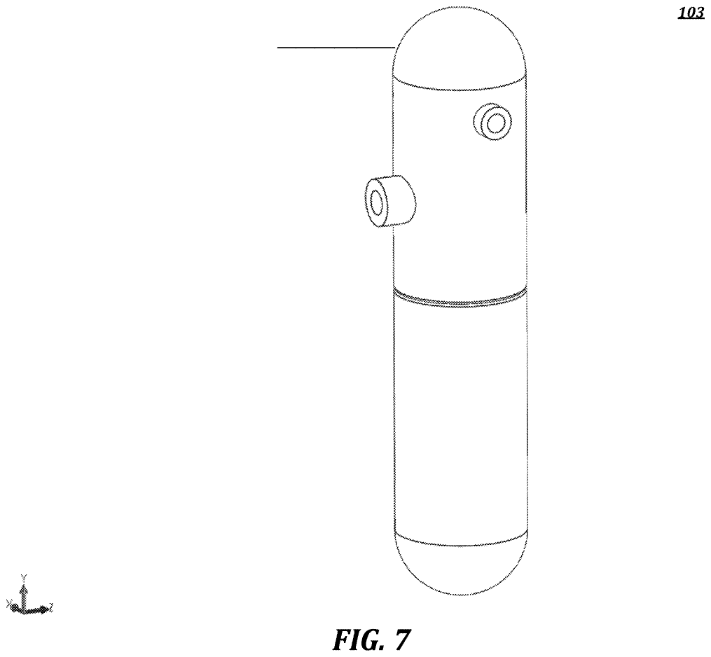

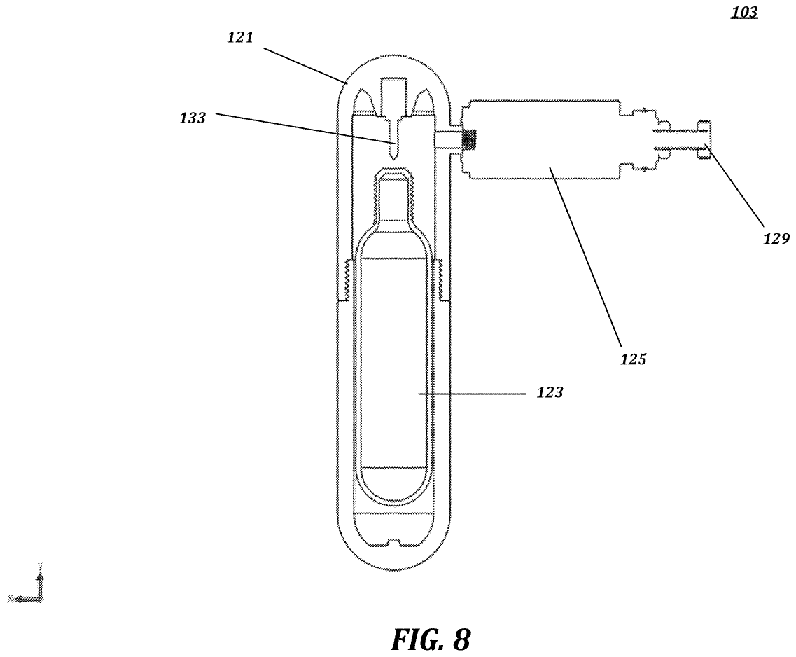

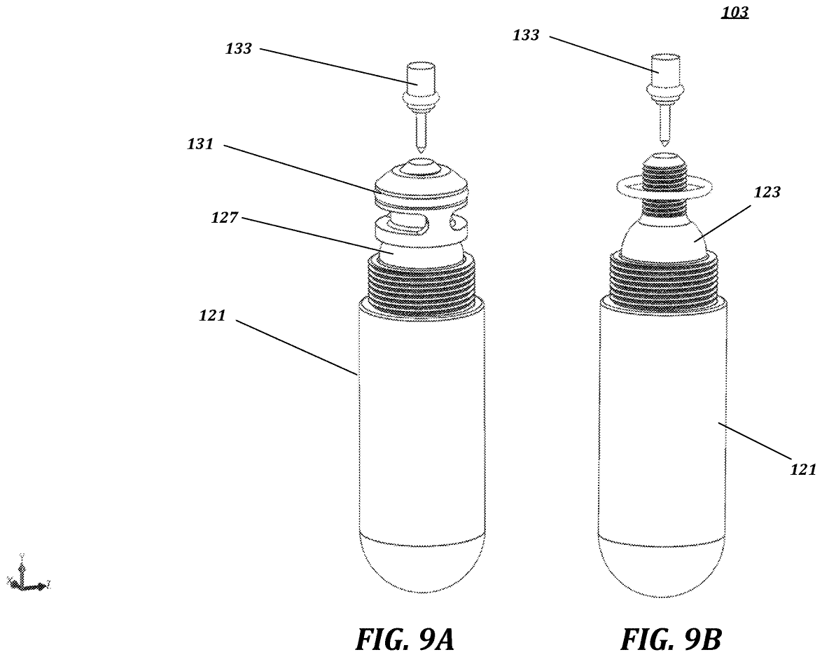

According to various further aspects of the disclosure, the tourniquet devices and systems can comprise multiple configurations. For example, various exemplary embodiments of the inventive fast acting tourniquet devices, systems, and methods are shown in FIGS. 1-13.

In one aspect, FIG. 1 depicts a device 100 for controlling blood flow to a body limb member of a user, the device comprising a band assembly 101 comprising: a band 102 with an air bladder 107 having a predetermined inflation volume and connected to the band and configured to provide a predetermined inflation level and/or inflation pressure and fastening strap 111 or other securing mechanism configured to releasably secure the band assembly and air bladder around a body limb member; and an air canister assembly 103 detachably connected to the band assembly, the air canister assembly comprising a pressurized gas source for inflating the air bladder. In an aspect, the band and air bladder cooperate to provide a medium through which pressure is applied to the body limb member.

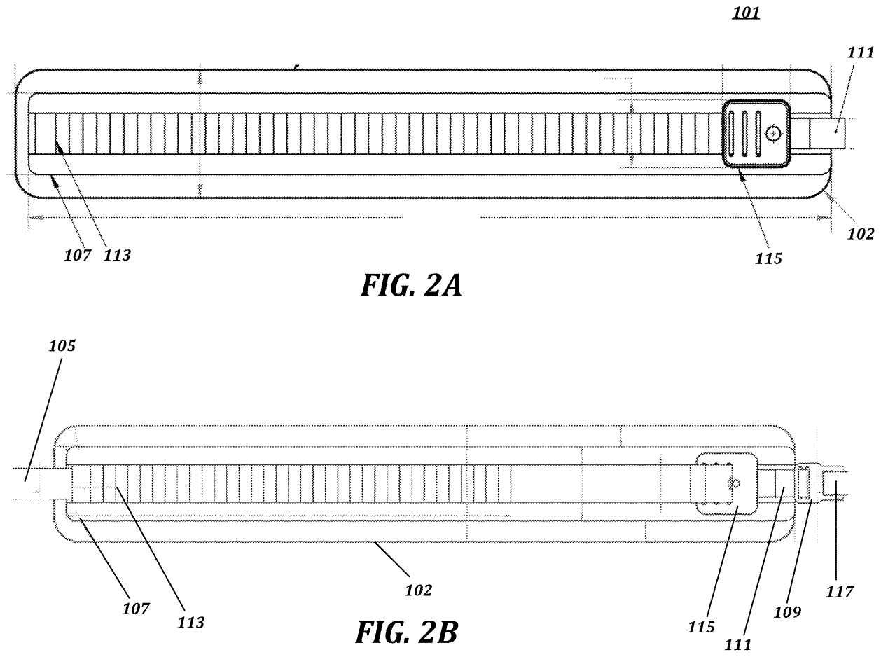

In further exemplary aspects, FIGS. 2A-4B and 14 show various depictions and components of a band assembly 101 in accordance with an embodiment of the present disclosure. As shown, the band assembly 101 includes a band 102 with an assistive slap band portion including restorably deformable member 105 having a one-way and/or directional potential energy driven state transition. Band 102 may include one or more layers, including a top layer 102a, a middle layer 102b, and a bottom layer 102c. In some embodiments, the top layer 102a may comprise a neoprene material, the middle layer 102b may comprise a textile or fabric, and/or the bottom layer 102c may comprise a nylon strip. The band assembly 101 may further include a latch 109 connected to band 102 using fastening strap 111. Latch 109 may be configured to attach to one of the plurality of loops 113, which are formed using overlapping webbing. Latch 109 may include a pull tab 117 to facilitate securing to loop 113. Band assembly 101 may further include an air canister interface panel 115 for coupling the air canister assembly to the band assembly 101. As shown, the air canister interface panel 115 may include an aperture to which the pressurized gas stream is delivered to for inflating the air bladder. Air canister interface panel 115 may include a strap or other fastener for releasably securing the air canister assembly 103.

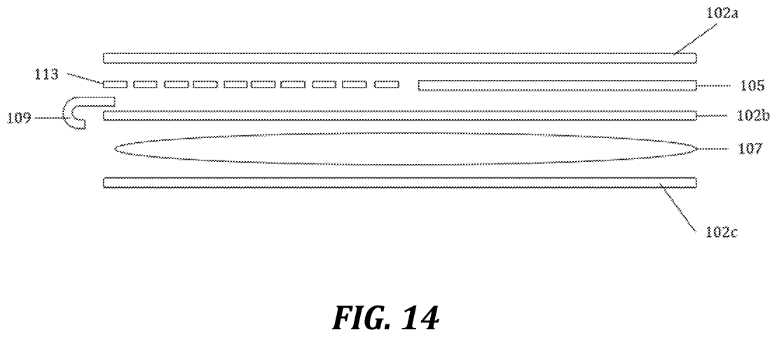

FIG. 14 may illustrate a cross-sectional view of the band assembly 101 showing the multi-layer construction and internal component arrangement. The top layer 102a may be positioned as the outermost layer of the band assembly 101. The top layer 102a may comprise a durable fabric material such as nylon ripstop or polyester canvas. The top layer 102a may provide abrasion resistance and protection for the internal components. The top layer 102a may have a thickness of approximately 0.005 to 0.020 inches. The material selection for the top layer 102a may prioritize tear resistance and puncture resistance to protect the internal air bladder during field use.

The spring steel encircling member 105 may be positioned between the top layer 102a and the middle layer 102b. The spring steel member 105 may comprise a flat spring steel strip with specific dimensional characteristics. The spring steel member 105 may have a thickness of approximately 0.010 to 0.030 inches. The spring steel member 105 may have a width of approximately 0.5 to 2.0 inches. The spring steel member 105 may extend along a portion of the band length, typically 8 to 16 inches depending on the intended limb circumference. The spring steel member 105 may be manufactured from high-carbon steel alloys such as 1074, 1084, or 1095 carbon steel. The spring steel member 105 may be heat-treated to achieve a hardness of 45-50 HRC on the Rockwell C scale.

The spring steel member 105 may be pre-stressed during manufacturing to create a natural coiled configuration. The resting diameter of the coiled configuration may be approximately 2 to 6 inches. The spring steel member 105 may store potential energy when held in the extended state. The stored elastic energy may drive the rapid transition from the extended state to the coiled state upon activation. The transition may occur in less than 0.5 seconds when the band is struck against a limb surface. The spring steel member 105 may be pre-formed with a slight helical twist along its length. The twist angle may be approximately 15 to 45 degrees over the length of the member. The helical twist may create a natural tendency to wrap in a spiral pattern rather than forming a simple circle.

The middle layer 102b may be positioned between the spring steel member 105 and the air bladder 107. The middle layer 102b may comprise a flexible separator material. The middle layer 102b may comprise thin polyurethane film, polyethylene sheet, or fabric membrane. The middle layer 102b may isolate the spring steel member 105 from the air bladder 107. The middle layer 102b may allow controlled flexing during deployment of the spring steel member. The middle layer 102b may have a thickness of approximately 0.003 to 0.015 inches. The middle layer 102b may provide a smooth surface for the spring steel member to slide against during coiling action.

The air bladder 107 may be positioned between the middle layer 102b and the bottom layer 102c. The air bladder 107 may comprise medical-grade vinyl, silicone, or reinforced plastic material. The air bladder 107 may have an elongated chamber configuration matching the band dimensions. The air bladder 107 may have a predetermined volume capacity of approximately 50 to 200 cubic centimeters depending on limb size. The air bladder 107 may be designed for operating pressures of 200 to 300 millimeters of mercury. The air bladder 107 may comprise flexible, expandable material with controlled stretch characteristics. The walls of the air bladder 107 may be heat-sealed or welded to create air-tight integrity. The seams of the air bladder 107 may be reinforced to prevent leakage under pressure.

The air bladder 107 may include an inlet port for connection to the gas supply line. The inlet port may be positioned at a predetermined location along the bladder length. The inlet port may comprise a threaded fitting, barbed connector, or quick-connect interface. The inlet port may be reinforced with a backing plate or grommet to prevent tearing of the bladder material. The air bladder 107 may include internal baffles or channels to promote uniform pressure distribution. The baffles may be formed by selective bonding of the bladder walls at predetermined locations. The baffle spacing may be approximately 0.5 to 2.0 inches. The baffles may create individual pressure cells that conform independently to limb contours.

The bottom layer 102c may be positioned as the innermost layer that contacts the user's skin or clothing. The bottom layer 102c may comprise a soft, biocompatible material. The bottom layer 102c may comprise closed-cell foam, fabric padding, or flexible polymer. The bottom layer 102c may have a thickness of approximately 0.020 to 0.100 inches. The bottom layer 102c may provide cushioning to distribute pressure evenly across the contact area. The bottom layer 102c may prevent pressure points that could cause tissue damage. The bottom layer 102c may include perforations or breathable construction to allow air circulation. The breathable construction may provide moisture management during extended wear periods.

The latch 109 may be positioned at a terminal end of the band assembly. The latch 109 may comprise a hook component of a hook-and-loop fastening system. The latch 109 may be attached to the band using stitching, adhesive bonding, or heat welding. The latch 109 may be configured to engage with loops 113 disposed on the outer surface of the band. The latch 109 may provide secure attachment while enabling rapid release under emergency conditions. The latch 109 may include a pull tab to facilitate one-handed manipulation. The pull tab may extend from the latch body to provide a gripping surface. The pull tab may be color-coded or textured to provide visual and tactile identification.

The loops 113 may be disposed on the outer surface of the top layer 102a. The loops 113 may comprise loop material of a hook-and-loop fastening system. The loops 113 may extend along a portion of the band length to allow adjustment for different limb sizes. The loops 113 may be arranged in multiple sections or as a continuous strip. The loops 113 may provide multiple engagement points for the latch 109. The hook-and-loop interface between the latch 109 and loops 113 may provide holding strength of approximately 5 to 15 pounds per square inch. The holding strength may be sufficient to maintain band position against pneumatic expansion forces. The hook-and-loop interface may allow manual release by pulling with approximately 10 to 25 pounds of force.

The multi-layer construction illustrated in FIG. 14 may provide structural integration of multiple functional elements. The layered arrangement may maintain flexibility while providing structural support. The layers may be bonded together using stitching, adhesive, or heat welding techniques. The bonding may create a unified band structure that functions as a single assembly. The bonding may allow relative movement between layers during deployment and inflation. The top layer 102a may protect the internal components from environmental damage. The spring steel member 105 may provide rapid deployment capability. The middle layer 102b may isolate mechanical components from pneumatic components. The air bladder 107 may provide controlled pressure application. The bottom layer 102c may provide comfort and pressure distribution.

The cross-sectional view of FIG. 14 may represent the band assembly in a flat, undeployed state. In this state, the spring steel member 105 may be held in an extended configuration. The air bladder 107 may be in a deflated state with minimal thickness. The overall thickness of the band assembly in the flat state may be approximately 0.25 to 0.75 inches. The flat configuration may allow compact storage and easy handling prior to deployment. The band assembly may be folded or rolled for storage in medical kits or tactical gear.

During deployment, the spring steel member 105 may transition from the extended state to the coiled state. The coiling action may cause the band assembly to wrap around the target limb. The layers may flex and conform to the limb contour during coiling. The bottom layer 102c may contact the limb surface first. The spring steel member 105 may provide circumferential tension that holds the band in position. The latch 109 may then be engaged with the loops 113 to secure the band. Following securement, the air bladder 107 may be inflated with pressurized gas. The inflation may cause the air bladder 107 to expand in thickness. The expansion may be constrained by the spring steel member 105 and the fastening mechanism. The constrained expansion may direct pressure inward toward the limb surface.

The operational sequence illustrated by the component arrangement in FIG. 14 may demonstrate the functional integration of the band assembly. The spring steel member 105 may provide initial positioning and constraint. The air bladder 107 may provide controlled pressure application. The fastening mechanism comprising latch 109 and loops 113 may maintain band position during inflation. The multi-layer construction may distribute forces and pressures across the band width. The distributed forces may reduce peak pressure points and improve user comfort. The integrated design may eliminate the need for separate positioning straps, external stiffening bands, and pressure distribution pads required in conventional pneumatic tourniquet systems.

The use case for the band assembly illustrated in FIG. 14 may involve application to an injured limb with active hemorrhage. A user may grasp the band assembly with one hand. The user may position the band assembly against the limb proximal to the wound site. The user may strike the band assembly against the limb to trigger deployment of the spring steel member 105. The spring steel member 105 may automatically coil around the limb in less than 0.5 seconds. The user may then wrap any remaining band length around the limb. The user may engage the latch 109 with the loops 113 using the same hand. The engagement may secure the band assembly in position. The user may then activate the gas release mechanism to inflate the air bladder 107. The inflation may occur within 2 to 5 seconds. The inflated air bladder 107 may apply circumferential pressure to the limb. The pressure may occlude arterial blood flow and stop hemorrhage.

Another use case may involve application by a first responder to an injured user. The responder may retrieve the band assembly from a medical kit. The responder may identify the optimal application location proximal to the injury. The responder may position the band assembly against the user's limb. The responder may activate the spring steel deployment by striking the band against the limb. The automatic coiling may eliminate the need for manual wrapping. The responder may secure the fastening mechanism with one hand. The responder may activate the inflation system with the same hand. The rapid application sequence may allow the responder to achieve hemostasis in less than 10 seconds. The consistent pressure delivery may ensure effective treatment regardless of the responder's physical strength or training level.

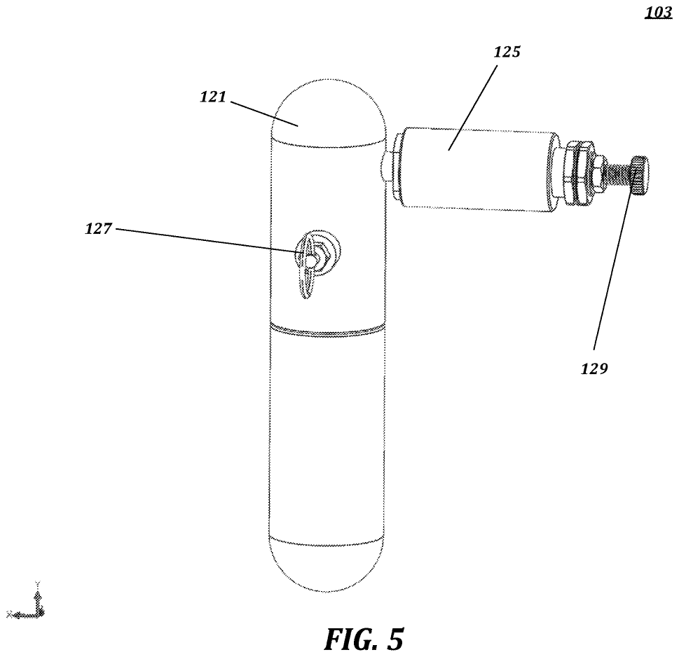

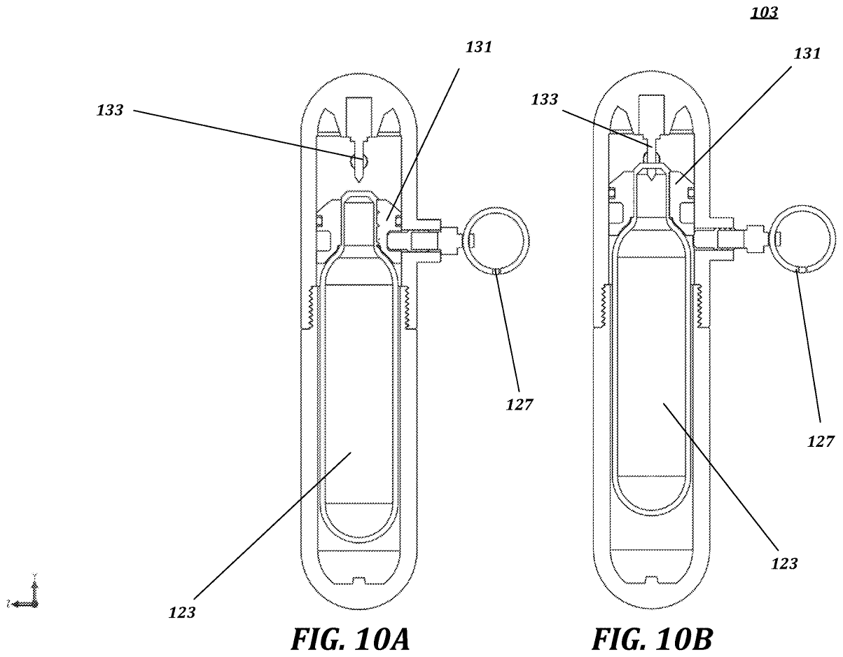

In further exemplary aspects, FIGS. 5-10B show various depictions and components of air canister assembly 103 in accordance with an embodiment of the present disclosure. Air canister assembly includes main housing 121 containing gas cartridge 123, air chamber 125 for containing pressurized air or gas therein prior to delivery to air bladder; activating pin 127 configured to activate inflation of the air bladder using the pressurized gas, and air valve with regulator 129 configured to control a rate or speed of inflation of the air bladder, or control an amount of pressure or force exerted by the device on the body limb member, or a combination thereof. Air chamber 125 includes an aperture configured to couple with the corresponding aperture on air canister assembly interface panel. As shown in FIGS. 10A-10B, removal of activating pin 127 activates tension mechanism 131, such as or spring element or other means for exerting a force against an end of the gas cartridge and/or puncture element 133, to cause the puncture element 133 to puncture gas cartridge to release gas.

The present disclosure, according to further aspects, also provides methods of using the disclosed devices and systems. In one aspect, disclosed herein is a method for using the disclosed fast-acting tourniquet devices and systems. A method may be provided for implementing a fast-acting tourniquet, comprising: applying a slap band of a tourniquet device to a limb member; securing the tourniquet device having an air bladder around the limb member using a latch; triggering inflation of the air bladder; and optionally controlling a release of the tourniquet device using an air valve. In various aspects, all the foregoing steps and methods of use may be configured to be completed using a single hand, whereby an injured user can self-administer the disclosed tourniquet devices.

In further aspects, the disclosed tourniquet devices and systems can be used to control or otherwise stop blood flow and/or bleeding. In an exemplary operation, a tourniquet may be configured to function in a particular manner. For example, initially a wound could be located. Upon identifying the wound, a user may slap on the slap band portion or segment of the inflatable band to secure an initial portion of the tourniquet device. The device uses the slap-band mechanism to initially secure the band to an area proximal to wound, such as approximately 1-6 inches or more above or below the wound. After securing the initial portion, the user can then wrap the remaining segments of the band and use the latch or fastening mean to secure in place around the limb. Subsequently thereafter, a user may pull a pull-tab to inflate an air bladder to a pre-designated pressure level or particular pounds per square inch (psi). The tourniquet device may be inflated to cause blood flow to stop, therefore not allowing the injured to bleed out. The present disclosure may be configured such that each aspect compliments another, but all may be paramount to achieve the goal of stopping the bleeding. As described herein, the slap band mechanism allows for full use and operation of the device using a single hand, which is critical in self-administration situations where the injured in limb is a limb belonging to the user.

In operation and use, a device of the present invention is effective in achieving the methods disclosed herein. In further aspects, the pressure from the band may be applied by the device to the selected area of the subject such that the band contacts the subject's limb. The inflated band be contacted with the surface for a time period for example, for a suitable time period so as to initiate blood flow control effects, for example, stopping blood flow to a wound or injury. The desired effect may depend on the method and technique of application of the pressure to the area. In still further aspects, after blood flow control effects are initiated, a treatment may occur to the subject or the occlusive pressure may be continued until all blood flow to the subject's limb is stopped. The occlusive pressure is applied to the subject for a time period sufficient to accomplish the effect intended in the disclosed methods, which can be between 0 seconds and several minutes or more depending on the subject and/or the method. For example, application and/or treatment time to the subject can be for a period of about 0 seconds to about 360 minutes, or longer in certain methods, to accomplish the effect intended in the disclosed methods. In further aspects, treatment times may comprise from about 0.5 minutes to about 2 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 20 minutes to about 30 minutes, from about 1 to 2 hours, from about 1 to about 3 hours, from about 2 to about 4 hours, and ranges therebetween. The treatment times listed here are not contemplated to be limiting to the methods of treatment of the invention. One of skill in the art can determine optimal treatment time periods. Also disclosed are various methods of using the disclosed devices to treat a user. For example, in another exemplary aspect, the present disclosure provides a method for controlling blood flow to a site on a user using a disclosed device.

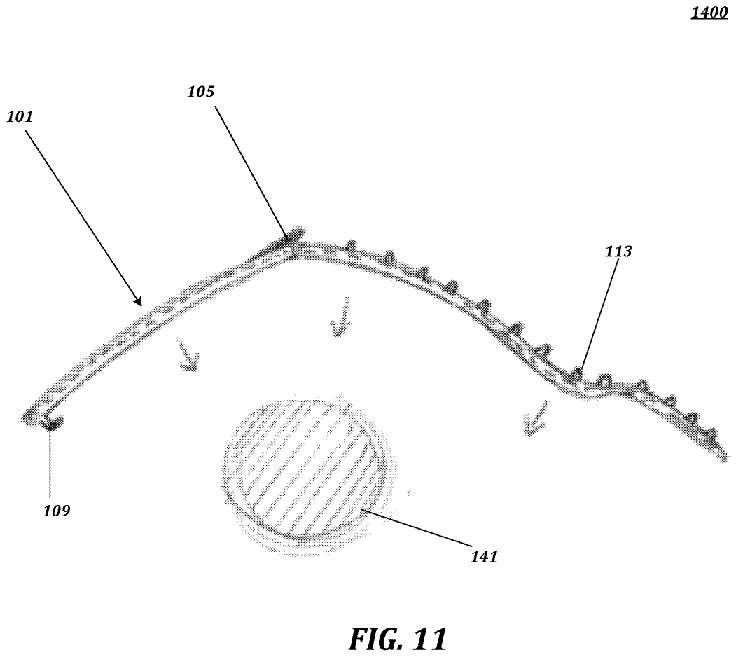

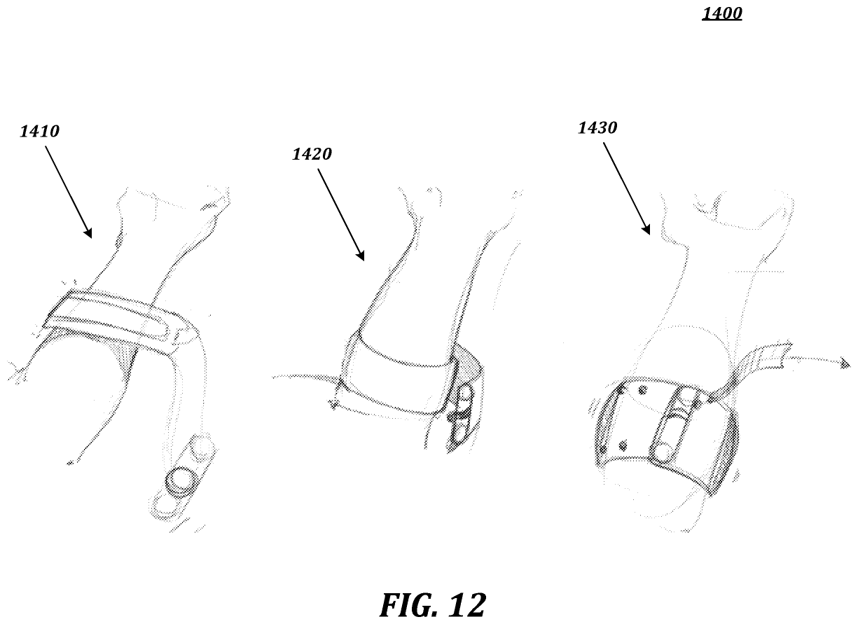

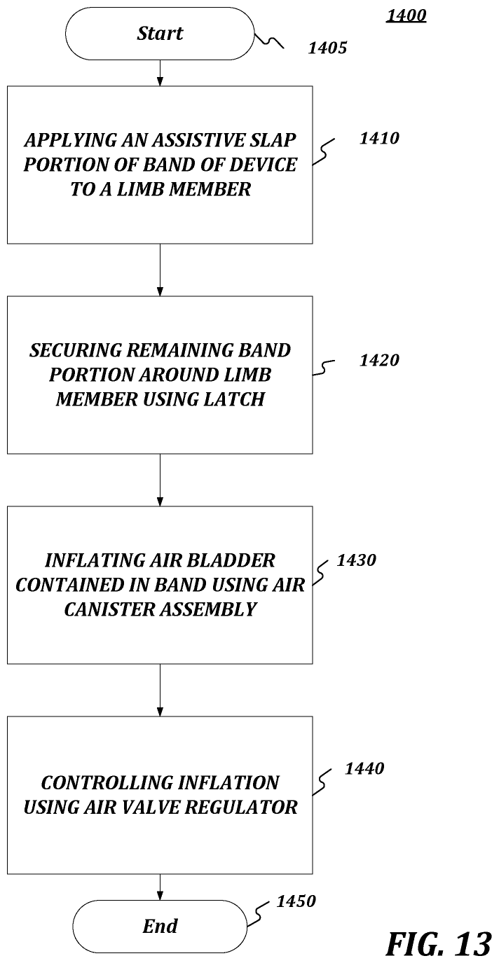

FIG. 13 show a flow chart, and FIGS. 11-12 show depictions setting forth the general stages involved in a method 1400 consistent with an embodiment of the disclosure for using a fast-acting tourniquet device, for example, to control or otherwise stop blood flow. Method 1400 may be implemented using any disclosed devices, such as, and without limitation, tourniquet device 100 as described herein. Although method 1400 has been described to be performed by tourniquet device 100, it should be understood that, in some embodiments, different operations may be performed by different elements directly coupled with tourniquet device 100. For example, a pneumatic apparatus and/or tourniquet device 100 may be employed in the performance of some or all of the stages in method 1400. Moreover, a pneumatic device may be configured much like tourniquet device 100 and, in some instances, be one and the same embodiment.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 1400 will be described in greater detail below.

Method 1400 may begin at starting block 1405 and proceed to stage 1410 where a user may apply the assistive slap band segment of the inflatable band of the device to a limb member 141. For example, a paramedic or a user may apply the slap band segment to an arm or leg of a bleeding user.

From stage 1410, where a user applied the assistive slap band segment of the device to a limb member, method 1400 may advance to stage 1420 where the remaining portion of the inflatable band can be wrapped around the limb and secured around the limb member using a latch or hook. For example, a user or a paramedic may secure the tourniquet device around an arm or leg of a bleeding user using a pull tab attached to the latch and inserting the latch in one of a plurality of loops disposed on the outer surface of the band.

Once tourniquet device is secured around the limb member using the latch in stage 1420, method 1400 may continue to stage 1430 where the user can trigger inflation of the air bladder using an air canister. For example, a user or a paramedic may trigger inflation of the tourniquet device by activating an activating switch or pull tab connected to an air canister.

After tourniquet device has been triggered using an air canister in stage 1430, method 1400 may proceed to stage 1440 where a level and/or rate of inflation may be controlled using an air valve regulator, depending on the clinical condition and specific user characteristics. For example, a user or paramedic may use the air valve regulator to regulate the release of pressurized gas or releasing pressure from the tourniquet device. After tourniquet device is left in place to control blood flow in 1440, method 1400 may then end at stage 1450 where the device can be removed.

The present invention includes at least the following aspects:

Aspect 1: A device for controlling local blood flow in a user, the device comprising: a pressurized gas source in operable communication with a switch configured to selectively release or dispense a predetermined volume of gas from the pressurized gas source in a gas stream having a predetermined flow rate and a predetermined flow path; and a band comprising a bendable encircling member, an air compartment or air bladder compartment, and a fastening mechanism or means configured to releasably secure the band around a portion of the user's body; the air compartment in fluid communication with the gas source or gas stream; the encircling member having an extended state and coiled state, the coiled state configured to circumferentially couple around a portion of a user's body; wherein the gas stream is configured to fill at least a portion of the air compartment effective to cause the band to exert a predetermined level of pressure against the portion of the user's body effective to reduce local blood flow.

Aspect 2: A device for controlling blood flow to a body limb member of a user, the device comprising: a band assembly comprising: a band with an assistive slap band portion including a restorably deformable member having a one-way and/or directional potential energy driven state transition; an air bladder having a predetermined inflation volume and connected to the band and configured to provide a predetermined inflation level and/or inflation pressure; and a latch or other securing mechanism configured to releasably secure the band assembly and air bladder around a body limb member; an air canister assembly detachably connected to the band assembly, the air canister assembly comprising: an air canister or air chamber containing pressurized air or gas therein; an activating element configured to activate inflation of the air bladder using the pressurized gas, and an air valve configured to control a rate or speed of inflation of the air bladder, or control an amount of pressure or force exerted by the device on the body limb member, or a combination thereof; wherein the band and air bladder cooperate to provide a medium through which pressure is applied to the body limb member.

Aspect 3: The device of any preceding aspect, wherein the encircling member comprises a restorably deformable member having a one-way and/or directional potential energy driven state transition.

Aspect 4: The device of any preceding aspect, wherein the encircling member comprises a spring mechanism or means for producing a coiling action, such as a spring steel.

Aspect 5: The device of any preceding aspect, wherein the encircling member is disposed within a first portion or segment of the band.

Aspect 6: The device of any preceding aspect, wherein the band comprises a plurality of different segments.

Aspect 7: The device of any preceding aspect, wherein the band comprises an assistive segment or portion containing the encircling member or spring mechanism and a wrapping segment without an encircling member.

Aspect 8: The device of any preceding aspect, wherein the band comprises an assistive segment containing the encircling member effective to assist with an initial clamping or encircling of the band around the body.

Aspect 9: The device of any preceding aspect, wherein the band comprises an assistive segment containing the encircling member effective to assist with an initial clamping or encircling of the assistive segment or portion of the band around the body.

Aspect 10: The device of any preceding aspect, wherein after the assistive segment is slapped, clamped, disposed around or otherwise secured against the body, another segment is wrapped around the body and secured to the outside surface of the band using the fastening means.

Aspect 11: The device of any preceding aspect, wherein the encircling member is configured to encircle around a limb upon striking or slapping it against the limb.

Aspect 12: The device of any preceding aspect, wherein the encircling member is configured to encircle around a limb upon striking or slapping an area of the band corresponding to the location of the member against the limb.

Aspect 13: The device of any preceding aspect, wherein the encircling member is configured to encircle around a limb upon striking or slapping it against the limb effective to cause it to go from an erect or extended state to a coiled state.

Aspect 14: The device of any preceding aspect, wherein the encircling member and/or assistive segment comprise a thumb tab or other protrusion for guiding and/or assisting with an initial securing step.

Aspect 15: The device of any preceding aspect, wherein the air compartment comprises an air bladder or chamber, or the like.

Aspect 16: The device of any preceding aspect, further comprising a housing or casing for housing one or more device components.

Aspect 17: The device of any preceding aspect, wherein the housing or casing is configured for containing a pressurized gas source or air canister.

Aspect 18: The device of any preceding aspect, further comprising at least one gas or air supply line configured to contain and/or carry the gas stream.

Aspect 19: The device of any preceding aspect, wherein the gas supply line is in fluid communication with the pressurized gas source.

Aspect 20: The device of any preceding aspect, wherein the gas supply line is in fluid communication with the air compartment or air bladder.

Aspect 21: The device of any preceding aspect, wherein the pressurized gas source comprises at least one of a pressurized gas vessel, canister, bottle, cylinder, tank, and cartridge, and the like.

Aspect 22: The device of any preceding aspect, wherein the pressurized gas source is contained with a device housing or air canister assembly.

Aspect 23: The device of any preceding aspect, wherein the pressurized gas source is detachably connected to the band or band assembly.

Aspect 24: The device of any preceding aspect, wherein the pressurized gas source comprises an air chamber comprising a compressed gas cartridge containing pressurized gas and puncture element for releasing the pressurized gas from the gas cartridge.

Aspect 25: The device of any preceding aspect, wherein an air chamber is configured to contain the gas cartridge, puncture element and gas released from the gas cartridge.

Aspect 26: The device of any preceding aspect, wherein the air chamber comprises a tension element, or spring element or other means for exerting a force against an end of the gas cartridge and/or puncture element effective to cause the puncture element to release gas from the gas cartridge.

Aspect 27: The device of any preceding aspect, wherein the air chamber comprises a housing having a plurality of portions for containing the puncture element, gas cartridge, and/or spring element.

Aspect 28: The device of any preceding aspect, wherein the chamber comprises a removable blocking element or obstructing means for mechanically obstructing the puncture element from puncturing the gas cartridge to release gas.

Aspect 29: The device of any preceding aspect, the puncture element is configured to puncture the gas cartridge to release gas upon removal of the obstructing means.

Aspect 30: The device of any preceding aspect, the puncture element cooperates with the spring element to puncture the gas cartridge to release gas upon removal of the obstructing means.

Aspect 31: The device of any preceding aspect, wherein the pressurized gas source is an external source remotely located from the device.

Aspect 32: The device of any preceding aspect, wherein the band is configured to connect to an external pneumatic system or air source effective to allow gradual device removal from user or to ease the user out of the device instead of cutting it off directly.

Aspect 33: The device of any preceding aspect, wherein the device comprises a switch configured to control and/or activate flow of gas from the pressurized gas source.

Aspect 34: The device of any preceding aspect, wherein the switch is configured to control the flow of gas flowing through a gas supply line in fluid communication with the pressurized gas source.

Aspect 35: The device of any preceding aspect, wherein the switch is configured to control gas flow by causing a valve having a plurality of valve positions to move from a closed valve position to an open valve position.

Aspect 36: The device of any preceding aspect, wherein the valve is configured to control a rate of gas release from the pressurized gas source.

Aspect 37: The device of any preceding aspect, wherein the valve is configured to be toggled between a first rate of gas release and a second, slower rate of gas release.

Aspect 38: The device of any preceding aspect, wherein the valve is in operative communication with a toggle for changing the rate of gas release.

Aspect 39: The device of any preceding aspect, wherein the valve is in operative communication with a toggle for changing the rate of gas release between a plurality of predetermined rates of gas release.

Aspect 40: The device of any preceding aspect, wherein the valve is in operative communication with a toggle for changing the rate of gas release between a first predetermined rate of gas release and a second predetermined rate of gas release.

Aspect 41: The device of any preceding aspect, wherein the valve is in operative communication with a toggling mechanism for cooperatively controlling a rate of inflation of the air bladder.

Aspect 42: The device of any preceding aspect, wherein the valve is in operative communication with a toggling mechanism for cooperatively controlling a rate of inflation of the air bladder from a first predetermined rate of inflation to a second predetermined rate of inflation.

Aspect 43: The device of any preceding aspect, wherein the toggle or toggle mechanism is user operated for changing rates.

Aspect 44: The device of any preceding aspect, wherein a switch is in operable communication with a puncture element for releasing pressurized gas from the pressurized gas source.

Aspect 45: The device of any preceding aspect, wherein a switch is in operable communication with a gas cartridge puncture element for releasing pressurized gas from a gas cartridge.

Aspect 46: The device of any preceding aspect, wherein the switch comprises a removable tab or barrier, and wherein the device may be activated by the removal of the tab or barrier.

Aspect 47: The device of any preceding aspect, wherein the switch comprises a removable tab or barrier between the gas source and a puncture element for releasing pressurized gas from the gas source.

Aspect 48: The device of any preceding aspect, wherein the switch comprises a removable tab or barrier between a gas cartridge and a gas cartridge puncture element for releasing pressurized gas from the gas cartridge.

Aspect 49: The device of any preceding aspect, wherein the switch comprises an actuating mechanism.

Aspect 50: The device of any preceding aspect, wherein the switch comprises an actuating mechanism connected to a puncture element.

Aspect 51: The device of any preceding aspect, wherein the switch comprises an actuating mechanism hingedly connected to a puncture element.

Aspect 52: The device of any preceding aspect, wherein the switch comprises an actuating mechanism hingedly connected to a puncture element and is configured to activate gas release from gas source by pulling on the switch.

Aspect 53: The device of any preceding aspect, wherein the switch comprises a toggle, lever, tab, pin, key, push-button, button-triggered valve, or the like.

Aspect 54: The device of any preceding aspect, wherein the switch is in operative communication with a valve for selectively causing a valve to move from at least one of: a closed position to an open position or an open position to a closed position to control gas flow from the gas source.

Aspect 55: The device of any preceding aspect, wherein the device is self-regulating.

Aspect 56: The device of any preceding aspect, further comprising a gas regulator or other means for controlling a gas flow rate and/or gas volume dispensed from the gas source.

Aspect 57: The device of any preceding aspect, further comprising a flow control component configured to control a gas flow rate and/or gas volume.

Aspect 58: The device of any preceding aspect, further comprising a flow control component in fluid communication with the gas stream, and configured to control a gas flow rate and/or gas volume from a gas stream.

Aspect 59: The device of any preceding aspect, wherein the flow control component is configured to control gas flow rate and/or volume entrance into the air compartment and/or exit from the air compartment.

Aspect 60: The device of any preceding aspect, further comprising a flow control component in fluid communication with the air compartment, and configured to control an entrance flow rate of gas and/or an entrance volume of gas.

Aspect 61: The device of any preceding aspect, further comprising a flow control component in fluid communication with the air compartment and configured to control release of gas from the air compartment.

Aspect 62: The device of any preceding aspect, further comprising a flow control component in fluid communication with the air compartment, and configured to control an exit flow rate and/or exit volume of gas.

Aspect 63: The device of any preceding aspect, wherein the fastening component or means comprises a hook and loop, hook and catch, latch, buckle, clip, snap, or the like.

Aspect 64: The device of any preceding aspect, wherein the fastening component or means is a latch, buckle, or the like.

Aspect 65: The device of any preceding aspect, wherein the fastening component or means comprises a cinching mechanism for securing the band around the portion of the user's body.

Aspect 66: The device of any preceding aspect, wherein the fastening component or means is a hook and loop arrangement.

Aspect 67: The device of any preceding aspect, wherein the fastening component or means is a hook connected a first end of the band and a plurality of loops disposed on an outer surface of the band.

Aspect 68: The device of any preceding aspect, wherein the band comprises a cuff, wrap or structural means for encircling a portion of a user's body or limb.

Aspect 69: The device of any preceding aspect, wherein the band is sized and dimensioned effective to releasably encircle a body limb, such as a thigh or arm.

Aspect 70: The device of any preceding aspect, wherein the band has a closed diameter of from about 1 in to about 36 inches, for example from about 12 inches to about 24 inches.

Aspect 71: The device of any preceding aspect, wherein the band has an opened length of from about 12 in to about 48 inches, for example from about 12 inches to about 36 inches.

Aspect 72: The device of any preceding aspect, wherein the band is comprised of neoprene or other soft material, or nylon and/or other tough and durable material, or combinations thereof.

Aspect 73: The device of any preceding aspect, wherein the valve comprises an air valve configured to control an inflation level of the air bladder by allowing a controlled release of gas or air, wherein the controlled release is at least one of consistent, intermittent, and variable.

Aspect 74: The device of any preceding aspect, wherein the air valve is further configured to control release of the device by allowing a controlled release of gas or air, wherein the controlled release is at least one of consistent, intermittent, and variable.

Aspect 75: The device of any preceding aspect, wherein the air valve is further configured to be directly attached to a pneumatic system wherein the direct attachment may control inflation level, such as by gradual deflation/inflation or gas/air release.

Aspect 76: A method for controlling local blood flow to a body limb or body part of a user, the method comprising the steps of: positioning a deformable slap band of a tourniquet device having an air bladder on and/or around a body limb or body part; securing the tourniquet device to the body limb or part using a fastening component or latch; and inflating the air bladder using pressurized gas contained within an air canister or chamber effective to control blood flow local to the body part or body limb.

Aspect 77: The method of any preceding aspect, further comprising controlling a rate of inflation or an inflation level of the air bladder using an air valve in fluid communication with the air bladder.

Aspect 78: The method of any preceding aspect, wherein all the method steps can be performed by a single hand of a user.

Aspect 79: The method of any preceding aspect, wherein all the method steps can be performed by a user on themselves using a single hand.

Aspect 80: The method of any preceding aspect, wherein control of local blood flow comprises complete block of blood flow.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order.

Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein can be different from the actual publication dates, which can require independent confirmation.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure. Insofar as the description above and the accompanying drawing disclose any additional subject matter that is not within the scope of the claims below, the disclosures are not dedicated to the public and the right to file one or more applications to claims such additional disclosures is reserved. Although very narrow claims are presented herein, it should be recognized that the scope of this disclosure is much broader than presented by the claims.

The following is claimed:

1. A pneumatic tourniquet system comprising:
a pressurized gas source containing compressed gas;
a pressure regulator configured to reduce gas pressure from the pressurized gas source to a working pressure;
an inflatable bladder configured to receive gas from the pressure regulator;
a gas delivery conduit providing fluid communication between the pressure regulator and the inflatable bladder;
a band assembly configured to position the inflatable bladder against a limb for hemostatic pressure application; and
a fastening mechanism configured to secure the band assembly around the limb;
wherein the pneumatic system delivers gas to the inflatable bladder to provide controlled arterial restriction.

2. The pneumatic tourniquet system of claim 1, wherein the pressurized gas source comprises a gas canister configured to provide multiple inflation cycles.

3. The pneumatic tourniquet system of claim 1, wherein the pressure regulator comprises a spring-loaded diaphragm mechanism that maintains consistent output pressure.

4. The pneumatic tourniquet system of claim 1, further comprising a dual-rate valve system configured to toggle between a first inflation rate and a second inflation rate, wherein neither rate equals zero.

5. The pneumatic tourniquet system of claim 1, wherein the band assembly includes a spring steel encircling member that provides structural constraint preventing the inflatable bladder from expanding radially outward during gas inflation.

6. The pneumatic tourniquet system of claim 5, wherein the spring steel encircling member has a one-way directional potential energy driven state transition from an extended state to a coiled state.

7. The pneumatic tourniquet system of claim 1, further comprising a pressure monitoring system configured to provide pressure readings during operation.

8. The pneumatic tourniquet system of claim 1, further comprising a pressure relief valve configured to automatically vent gas when bladder pressure exceeds a predetermined maximum pressure.

9. The pneumatic tourniquet system of claim 1, further comprising an emergency deflation system configured to provide rapid bladder deflation through a vent port.

10. A multi-layer band assembly for a tourniquet device comprising:
a top layer configured to provide structural protection;
a middle layer disposed beneath the top layer;
a spring steel encircling member positioned between the top layer and middle layer;
a bottom layer disposed beneath the middle layer;
an inflatable chamber positioned between the middle layer and bottom layer; and
a fastening component configured to secure the band assembly around a limb;
wherein the spring steel encircling member has a one-way directional potential energy driven state transition from an extended state to a coiled state.

11. The multi-layer band assembly of claim 10, wherein the spring steel encircling member is configured to wrap around a limb in a helical pattern during the state transition.

12. The multi-layer band assembly of claim 10, wherein the inflatable chamber is sealed between the middle layer and bottom layer through bonded seams.

13. The multi-layer band assembly of claim 10, wherein the spring steel encircling member stores potential energy in an extended configuration and releases the energy during transition to the coiled state upon contact with a limb surface.

14. The multi-layer band assembly of claim 10, wherein the fastening component comprises a hook-and-loop system configured to provide secure attachment while allowing manual release.

15. The multi-layer band assembly of claim 10, wherein the inflatable chamber includes internal structure configured to promote uniform pressure distribution across a contact area.

16. An integrated pneumatic-mechanical tourniquet system comprising:
a pressurized gas source containing compressed gas;
a pressure regulator configured to reduce gas pressure from the pressurized gas source;
a multi-layer band assembly including a spring steel encircling member and an inflatable bladder; and
a gas delivery system providing fluid communication between the pressure regulator and the inflatable bladder;
wherein the spring steel encircling member provides structural constraint that prevents radial bladder expansion during pneumatic inflation, directing gas pressure inward for vascular compression.

17. The integrated system of claim 16, wherein the spring steel member maintains circumferential constraint as the inflatable bladder expands, enabling the pneumatic system to achieve effective hemostatic pressures with reduced gas volume requirements.

18. The integrated system of claim 16, wherein the spring steel member provides automatic positioning that optimizes pneumatic bladder placement for arterial compression, with mechanical coiling action ensuring consistent bladder-to-limb contact.

19. The integrated system of claim 16, wherein the multi-layer construction isolates the spring steel member from the inflatable bladder, preventing damage to the pneumatic system while allowing mechanical constraint forces to enhance pressure distribution.

20. The integrated system of claim 16, wherein the combination of pneumatic inflation and spring steel constraint maintains consistent vascular compression during physiological variations, with the spring steel member providing elastic response to tissue volume changes while the pneumatic system maintains baseline pressure.
